SEQUENCE LISTING (30 sequences)

SEQ ID NO: 1 - protein, 39 aa, Homo sapiens
  Y receptor agonist
YPSKPKDNPGEKDAPAEKDMARYTSALRHYINLITRQRY

SEQ ID NO: 2 - protein, 37 aa, Homo sapiens
  Y receptor agonist
TPIKPEAPGEKDASPEELNRYTASLRHYLNLVTRQRY

SEQ ID NO: 3 - protein, 38 aa, Homo sapiens
  Y receptor agonist
APLEPVYPGKDNATPEQMAQYAAKDLRRYINMLTRPRY

SEQ ID NO: 4 - protein, 38 aa, Artificial Sequence
  Y receptor agonist
APLEPVYPGKDNATPEQMAQYAAKDLRRYINTLTRQRY

SEQ ID NO: 5 - protein, 37 aa, Artificial Sequence
  Y receptor agonist
PLEPVYPGKDNATPEQMAQYAAKDLRRYINTLTRQRY

SEQ ID NO: 6 - protein, 38 aa, Artificial Sequence
  Y receptor agonist
APLEPVYPGKDNATPEQMAQYAAKDLRRYINTITRQRY

SEQ ID NO: 7 - protein, 37 aa, Artificial Sequence
  Y receptor agonist
PLEPVYPGKDNATPEQMAQYAAKDLRRYINTITRQRY

SEQ ID NO: 8 - protein, 36 aa, Artificial Sequence
  Y receptor agonist
PLEPVYPGENATPEQMAQYAAKDLRRYINTITRQRY

SEQ ID NO: 9 - protein, 37 aa, Artificial Sequence
  Y receptor agonist
PLEPVYPGKDNATPEQMAQYAAKDLRRYINMITRQRY

SEQ ID NO: 10 - protein, 37 aa, Artificial Sequence
  Y receptor agonist
YPIKPEAPGEKDASPEELNRYTASLRHYLNLVTRQRY

SEQ ID NO: 11 - protein, 38 aa, Artificial Sequence
  Y receptor agonist
APLEPVYPGKDNATPEQMAQYAAKDLRRYINMLTRQRY

SEQ ID NO: 12 - protein, 36 aa, Artificial Sequence
  Y receptor agonist
LEPVYPGKDNATPEQMAQYAAKDLRRYINMLTRQRY

SEQ ID NO: 13 - protein, 38 aa, Artificial Sequence
  Y receptor agonist
APVEPVYPGKDNATPEQMAQYAAKDLRRYINMLTRQRY

SEQ ID NO: 14 - protein, 38 aa, Artificial Sequence
  Y receptor agonist
APLEPVYPGKDNATPEQMAQYAAKDLRRYINMITRQRY

SEQ ID NO: 15 - protein, 38 aa, Artificial Sequence; identical to SEQ ID NO: 14
  Y receptor agonist

SEQ ID NO: 16 - protein, 38 aa, Artificial Sequence
  Y receptor agonist
APLEPVYPGKDNATPEQMAQYAAKDLRHYINMLTRQRY

SEQ ID NO: 17 - protein, 37 aa, Artificial Sequence
  Y receptor agonist
APIEPEAPGEKDASPEELNRYTASLRRYLNMVTRQRY

SEQ ID NO: 18 - protein, 37 aa, Artificial Sequence
  Y receptor agonist
PLEPVYPGKDNATPEQMAQYAAKDLRRYINMLTRQRY

SEQ ID NO: 19 - protein, 6 aa, Artificial Sequence
  GAG motif
XXXXXX

SEQ ID NO: 20 - protein, 8 aa, Artificial Sequence
  GAG motif
XXXXXXXX

SEQ ID NO: 21 - protein, 16 aa, Artificial Sequence
  concatameric GAG motifs
XXXXXXXXXXXXXXXX

SEQ ID NO: 22 - protein, 16 aa, Artificial Sequence
  concatameric GAG motifs
ARRRAARAARRRAARA

SEQ ID NO: 23 - protein, 24 aa, Artificial Sequence
  concatameric GAG motifs
ARRRAARAARRRAARAARRRAARA

SEQ ID NO: 24 - protein, 16 aa, Artificial Sequence
  concatameric GAG motifs
XXXXXXXXXXXXXXXX

SEQ ID NO: 25 - protein, 24 aa, Artificial Sequence
  concatameric GAG motifs
XXXXXXXXXXXXXXXXXXXXXXXX

SEQ ID NO: 26 - protein, 24 aa, Artificial Sequence
  concatameric GAG motifs
ARRRAARAARRRAARAARRRAARA

SEQ ID NO: 27 - protein, 8 aa, Artificial Sequence
  GAG motif
ARRRAARA

SEQ ID NO: 28 - protein, 24 aa, Artificial Sequence
  concatameric GAG motifs
ARRRAARAARRRAARAARRRAARA

SEQ ID NO: 29 - protein, 27 aa, Artificial Sequence
  concatameric GAG motifs
ARRRAAARAARRRAAARAARRRAAARA

SEQ ID NO: 30 - protein, 6 aa, Artificial Sequence
  N terminal peptide attachment
KKKKKK

(19) United States
(12) Patent Application Publication
(10) Pub. No.: US 2010/0160226 A1
(43) Pub. Date: Jun. 24, 2010

(54) Y-RECEPTOR AGONISTS

(75) Inventors: Thue Schwartz, Hoersholm (DK); Christian Elling, Hoersholm (DK); Paul Brian Little, Hoersholm (DK); Anders Heding, Hoersholm (DK); Lars-Ole Gerlach, Hoersholm (DK)

Correspondence Address: BANNER & WITCOFF, LTD., 1100 13th STREET, N.W., SUITE 1200, WASHINGTON, DC, 20005-4051, US

(73) Assignee: 7TM PHARMA A/S, Hoersholm (DK)

(21) Appl. No.: 12/597,090

(22) PCT Filed: Apr. 21, 2008
(86) PCT No.: PCT/GB2008/001379

§ 371 Date: Feb. 26, 2010

(30) Foreign Application Priority Data
Apr. 27, 2007 (GB) 0708226.6

(51) Int. Cl. A61K 38/16; C07K 14/00; A61P 3/04; A61P 3/10; A61P 3/06

(52) U.S. Cl. 514/12; 530/324

(57) ABSTRACT

Human Pancreatic Polypeptide mutants are provided which have utility, inter alia, for regulation of energy intake or energy metabolism, control of intestinal secretion, decrease of gastrointestinal tract motility, decrease of rate of gastric emptying, treatment of obesity or overweight, or conditions in which obesity or overweight is a contributory factor.

This invention relates to peptide compounds that act as selective agonists of the Y2 and Y4 relative to the Y1 receptors, and to their use in treatment of conditions responsive to the activation of Y2 and/or Y4 receptors, for example treatment of obesity and overweight, and conditions in which these are considered contributory factors, and for controlling/decreasing GI-tract secretion

### BACKGROUND TO THE INVENTION

The PP-fold family of peptides—NPY (Neuropeptide Y) (human sequence—SEQ ID. No:1), PYY (Peptide YY) (human sequence SEQ ID. No:2), and PP (Pancreatic Polypeptide) (human sequence—SEQ ID. No:3), are naturally secreted homologous, 36 amino acid, C-terminally amidated peptides, which are characterized by a common three-dimensional, structure—the PP-fold—which is surprisingly stable even in dilute aqueous solution and is important for the receptor recognition of the peptides.

NPY is a very wide-spread neuropeptide with multiple actions in various parts of both the central and peripheral nervous system acting through a number of different receptor subtypes in man: Y1, Y2, Y4 and Y5. The main NPY receptors are the Y1 receptor, which is the post-synaptic receptor conveying the “action” of the NPY neurons, and the Y2 receptor which generally is a pre-synaptic, inhibitory receptor. This is also the case in the hypothalamus, where NPY neurones—which also express the melanocortin receptor antagonist/inverse agonist AgRP (agouti related peptide)—act as the primary “sensory” neurones in the stimulatory branch of the arcuate nucleus. Thus, in this the “sensor nucleus” for the control of appetite and energy expenditure, the NPY/AgRP neurones together with the inhibitory POMC/CART neurones monitor the hormonal and nutritional status of the body as these neurones are the target for both the long-term regulators such as leptin and insulin and short term regulators such as ghrelin and PYY (see below). The stimulatory NPY/AgRP neurones project for example to the paraventricular nucleus—also of the hypothalamus—where its postsynaptic target receptors are believed to be Y1 and Y5 receptors. NPY is the most potent compound known in respect of increasing food intake, as rodents upon intracerebroventricular (ICV) injection of NPY will eat until they literally burst. AgRP from the NPY/AgRP neurones acts as an antagonist mainly on melanocortin receptors type 4 (MC-4) and block the action of POMC derived peptides—mainly aMSH—on this receptor. Since the MC4 receptor signal acts as an inhibitor of food intake, the action of AgRP is—just like the NPY action—a stimulatory signal for food intake (i.e. an inhibition of an inhibition). On the NPY/AGRP neurons are found inhibitory—pre-synaptic—Y2 receptors, which are the target both of locally released NPY as well as a target for the gut hormone PYY—another PP-fold peptide.

PYY is released during a meal—in proportion to the calorie content of the meal—from entero-endocrine cells in the distal small intestine and the colon, to act both in the periphery on GI-tract functions and centrally as a satiety signal. Peripherally, PYY is believed to function as an inhibitor—an “illeal break”—on for example upper GI-tract motility, gastric acid and exocrine pancreatic secretion. Centrally, PYY is believed to act mainly on the presynaptic, inhibitory Y2 receptors on the NPY/AgRP neurones in the arcuate nucleus, which it is believed to get access to from the blood (Batterham et al. 2002 Nature 418: 650-4). The peptide is released as PYY1-36, but a fraction—approximately 50%—circulates as PYY3-36 which is a product of degradation by dipeptidylpeptidase-IV an enzyme which removes a dipeptide from the N-terminus of a peptide provided that a Pro or Ala is found in position two as in all three PP-fold peptides—PP, PYY and NPY (Eberlein et al. 1989 Peptides 10: 797-803). Thus PYY in the circulation is a mixture of PYY1-36, which acts on both Y1 and Y2 receptors (as well as Y4 and Y5 with various affinities), and PYY3-36—which has lower affinities for the Y1, Y4 and Y5 receptors than for the Y2 receptor.

PP is a hormone, which is released from endocrine cells in the pancreatic islets, almost exclusively governed by vagal cholinergic stimuli elicited by especially food intake (Schwartz 1983 Gastroenterology 85:1411-25). PP has various effects on the gastrointestinal tract, but these are generally not observed in isolated cells and organs, and appear to be dependent on an intact vagal nerve supply (Schwartz 1983 Gastroenterology 85:1411-25). In accordance with this, the PP receptors, which are called Y4 receptors, are located mainly in area postrema in the brain stem with a strong expression in vagal motor neurones—activation of which results in the peripheral effects of PP—and in the nucleus tractus solitarirus (NTS)—activation of which results in the effects of PP as a satiety hormone (Whitecomb et al. 1990 Am. J. Physiol. 259: G687-91, Larsen & Kristensen 1997 Brain Res. Mol. Brain Res 48: 1-6). It should be noted that PP from the blood has access to this area of the brain since the blood brain barrier is “leaky” in this area where various hormones from the periphery are sensed. Recently it has been argued that part of the effect of PP on food intake is mediated through an action on neurone's especially the POMC/CART neurones in the arcuate nucleus (Batterham et al. 2004 Abstract 3.3 International NPY Symposium in Coimbra, Portugal). PP acts through Y4 receptors for which it has a subnanomolar affinity as opposed to PYY and NPY which have nanomolar affinity for this receptor (Michel et al. 1998 Pharmacol. Rev. 50: 143-150). PP also has an appreciable affinity for the Y5 receptor, but it is not likely of physiological importance in relation to circulating PP due to both lack of access to the cells in the CNS where this receptor especially is expressed and due to the relatively low affinity for PP.

PP-fold peptide receptors—There are four well established types of PP-fold peptide receptors in man: Y1, Y2, Y4, and Y5 which all recognize NPY1-36 and PYY1-36 with similar affinity. At one time a Y3 receptor type, which might prefer NPY over PYY, was suggested, but today this is not accepted as a real receptor subtype (Michel et al. 1998 Pharmacol. Rev. 50: 143-150). A Y6 receptor subtype has been cloned, which in man is expressed in a truncated form lacking TM-VII as well as the receptor tail and consequently at least on its own does not appear to form a functional receptor molecule.

Y1 receptors—affinity studies suggest Y1 binds NPY and PYY equally well and basically not PP. Affinity for Y1 is dependent on the identities of both end sequences of the PP-fold molecule (NPY/PYY)—for example residues Tyr1 and Pro2 are essential—and it is dependent on the peptide ends being presented in just the right way. In the C-terminal end, where the side-chains of several of the residues are essential, the Y1 receptor—like the Y5 and Y4 receptor but not the Y2 receptor—tolerates certain substitutions in position 34 (normally a Gln)—such as Pro (Fuhlendorff et al. 1990 J. Biol. Chem. 265: 11706-12, Schwartz et al. 1990 Annals NY Acad. Sci. 61: 35-47). Some structure-function studies concerning the requirements of the Y1 and Y2 receptors have been reported (Beck-Sickinger et al. 1994 Eur. J. Biochem. 225: 947-58; Beck-Sickinger and Jung 1995 Biopolymers 37: 123-42; Söll et al. 2001 Eur. J. Biochem. 268: 2828-37).

Y2 receptors—affinity studies suggest Y2 binds NPY and PYY equally well and basically not PP. The receptor requires especially the C-terminal end of the PP-fold peptide (NPY/PYY). Thus, long C-terminal fragments—down to for example NPY13-36 (the whole alpha helix plus the C-terminal hexapeptide)—are recognized with relatively high affinity, i.e. to within ten-fold of the affinity of the full-length peptide (Sheikh et al. 1989 FEBS Lett. 245: 209-14, Sheikh et al. 1989 J. Biol. Chem. 264: 6648-54). Therefore various N-terminal deletions, which eliminate the binding to the Y1 receptor, still preserve some degree of binding to the Y2 receptor. However, the affinity of the C-terminal fragments is reduced-approximately 10 fold as compared to NPY/PYY for even relatively long fragments. The Gln residue in position 34 of NPY and PYY is highly important for the ligand recognition of the Y2 receptor (Schwartz et al. 1990 Annals NY Acad. Sci. 611: 35-47).

Y4 receptors—affinity studies suggest that Y4 binds PP with subnanomolar affinity corresponding to the concentrations found in plasma whereas NPY and PYY are recognized with much lower affinity. Such studies suggest the Y4 receptor is highly dependent on the C-terminal end of the PP-fold peptides, and that relatively short N-terminal deletions impairs the affinities of the ligands. Some structure activity studies concerning the Y4 receptor have been reported (Gehlert et al. 1996 Mol. Pharmacol. 50: 112-18; Walker et al. 1997 Peptides 18: 609-12).

Pharmaceutical use of PP-fold peptides—some PP-fold peptides have been suggested for use in the treatment of obesity and associated diseases, including for example Prader Willi's syndrome, based on the demonstrated effects of certain of the these peptides in animal models and in man and on the fact that obese people have low basal levels of PYY and PP as well as lower meal responses of these peptides (Hoist J J et al. 1983 Int. J. Obes. 7: 529-38; Batterham et al. 1990 Nature). Infusion of PP in patients with Prader Willi's syndrome was early on shown to decrease food intake (Berntson et al. 1993 Peptides 14: 497-503) and this effect has been confirmed by infusion of PP in normal human subjects (Batterham et al 2003, Clin. Endocrinol. Metab. 88: 3989-92). PP-fold peptides have also been suggested for the use in for example therapeutic angiogenesis (Zukowska et al. 2003 Trends Cardiovasc Med. 13:86-92) and in inflammatory bowl disease (see for example WO 03/105763).

However, the native PP-fold peptides are not optimal for use as biopharmaceuticals. For example, the full length peptides, PYY1-36 and NPY1-36 react too broadly with all Y receptor types and will therefore cause cardiovascular side effects and, for example, emesis. For the treatment of conditions responsive to Y receptor modulation, it would therefore be desirable to use Y receptor PP-fold peptides or PP-fold peptide mimics which were specific for the selected Y receptor intended as target.

Published International Patent Application WO 2005/089790 teaches the benefits of use of Y-receptor agonist peptides which are selective for the Y2 and Y4 receptors over the Y1 receptor. As explained in that publication, in several conditions, such as obesity and secretory diarrhoea, the use of an agonist at both the Y2 and Y4 receptors, but with low agonist activity at the Y1 receptor, is beneficial. Both the Y2 receptor and the Y4 receptor are potential targets for anti-obesity agents based on the notion that both the endogenous selective agonist for the Y2 receptor (PYY3-36), and for the Y4 receptor (PP), act as natural satiety hormones from the GI-tract to the CNS. Combined, selective Y2-Y4 agonists would, according to WO 2005/089790, have high potency at both the Y2 and the Y4 receptor but display low potency for the Y1 receptor, which is know to provide risk of cardiovascular and renal side effects.

WO 2005/089790 therefore disclosed a structural “algorithm” for generation of stable-PP-fold selective Y2-Y4 agonists, along with a number of specific peptides having this property. Two structural series of peptides were disclosed therein as being combined, selective Y2-Y4 agonist peptides: one which could be considered to be based on the PP peptide scaffold and another which could be considered to be based on the PYY scaffold.

Y5 receptors—affinity studies suggest that Y5 binds NPY and PYY equally well, and also binds PP with lower affinity, which however is below the normal circulating levels of this hormone. PYY3-36 is also recognized well by the Y5 receptor, however this receptor is to a large degree expressed in the CNS where such peptide cannot get access to the receptor readily when administered in the periphery.

The Y5 receptor was originally described as the NPY receptor which in the hypothalamus was responsible for the highly potent and efficacious stimulatory effect of centrally administered NPY on food intake. The Y5 receptor was for a time before and after its cloning also called the NPY “feeding receptor”. Subsequently, it became clear that NPY released from the NPY/AGRP neurons of the arcuate nucleus of the hypothalamus probably acts through both the Y1 and the Y5 receptors. In the periphery, the function of the Y5 receptor has been more unclear. However, in relation to the cardiovascular system the Y5 receptor has been described as a receptor which, when co-expressed in the periphery with either the Y1 or the Y2 receptor serves as an important helping receptor which provides increased activity to either the Y1 or the Y2 receptor, depending on the cellular setting and which receptor the Y5 receptor is co-expressed with. The molecular and/or cellular basis for this is unclear, but it has for example been suggested that the Y5 receptor may exerts its “helping” function through a hetero-dimeric setting.

Although WO 2005/089790 emphasised the benefits of combined selective Y2-Y4 versus Y1 peptide agonists, and some of the specific peptides disclosed therein had a some activity at the Y5 receptor, the structural algorithm disclosed in WO 2005/089790 for the design of combined Y2-Y4 agonists took no account of the level of Y5 agonist activity which was or would be possessed by the resultant peptide.

### BRIEF DESCRIPTION OF THE INVENTION

The present invention makes available specific peptides not disclosed per se in WO 2005/089790, and believed to be novel in the general art of PP-fold peptides, and which are combined selective Y2-Y4 versus Y1 agonists. The novel peptides of the invention have beneficially modified Y receptor agonist profiles relative to those particularized in WO 2005/089790.

### DETAILED DESCRIPTION OF THE INVENTION

According to the invention, there is provided a peptide selected from the group consisting of

(SEQ ID No: 4)

(1) [Thr30, Gln34]hPP,

(SEQ ID No: 5)

(2) [Thr30, Gln34]hPP2-36;

(SEQ ID No: 6)

(3) [Thr30, Ile31, Gln34]hPP;

(SEQ ID No: 7)

(4) [Thr30, Ile31, Gln34]hPP2-36;

(SEQ ID No: 8)

(5) [Glu10, Thr30, Ile31, Gln34]hPP2-36;
and

(SEQ ID No: 9)

(6) [Ile31, Gln34]hPP2-36;

(SEQ ID No: 18)

(7) [Gln34]hPP2-36;

and analogues thereof which are
(a) conservatively substituted in one or more positions other than position 34 in cases (1) to (7), and position 30 in cases (1) to (5), and position 31 cases (3) to (6) and position 10 in case (5); and/or
(b) N-terminally acylated, PEGylated, or covalently coupled to a serum albumin binding motif, a glycosaminoglycan binding motif or a helix inducing motif, said covalent coupling being to a residue of the peptide or to a residue substituted in peptide which provides a functional group for such covalent binding.

The notation hPP used herein refers to the human PP sequence (SEQ ID No: 3). Thus [Thr30,Gln34]hPP (SEQ ID No: 4) has the human PP sequence (SEQ ID No: 3) but with glutamine substituted for proline at position 34, and threonine substituted for methionine at position 30 thereof.

The notation hPP2-36 used herein refers to the human PP sequence (SEQ ID No:3) but with the first N-terminal amino acid (Ala) deleted. However, the position numbering of hPP2-36 is by reference to the full length hPP (SEQ ID No:3). Thus, the peptide [Thr30,Gln34]hPP2-36 (SEQ ID No: 5) has the human PP sequence SEQ ID No:3, but with Ala1 deleted, with glutamine substituted for proline at position 34 of SEQ ID No:3, and with threonine substituted for methionine at position 30 of SEQ ID No:3.

In this specification, reference is made to amino acids by their common names or abbreviations, such as valine (Val), leucine (Leu), isoleucine (Ile), methionine (Met), phenylalanine (Phe), asparagine (Asn), glutamic acid (Glu), glutamine (Gln), histidine (His), lysine (Lys), arginine (Arg), aspartic acid (Asp), glycine (Gly), alanine (Ala), serine (Ser), threonine (Thr), tyrosine (Tyr), tryptophane (Trp), cysteine (Cys) and proline (Pro). When referred to by its common name or abbreviation, without specifying its steroisomeric form, the amino acid in question is to be understood as the L-form.

The term “conservative substitution” as used herein denotes that one or more amino acids is replaced by another, biologically similar residue. Examples include substitution of amino acid residues with similar characteristics, e.g. small amino acids, acidic amino acids, polar amino acids, basic amino acids, hydrophobic amino acids and aromatic amino acids. Non-limiting examples of conservative amino acid substitutions suitable for use in the present invention include those in the following Table and analogous substitutions of the original residue by non-natural alpha amino acids which have similar, characteristics. For example, Met residues may be substituted with norleucine (Nle) which is a bioisostere for Met, but which—as opposed to Met—is not readily oxidised. Another example of a conservative substitution with a residue normally not found in endogenous, mammalian peptides and proteins is the conservative substitution of Arg or Lys with for example, ornithine, canavanine, aminoethylcysteine or other basic amino acid. For further information concerning phenotypically silent substitutions in peptides and proteins, see, for example, Bowie et. al. Science 247, 1306-1310, 1990.

| Original residue | Conservative substitution |
|---|---|
| Ala | Gly |
| Arg | Lys |
| Asn | Gln, His, Thr |
| Asp | Glu |
| Gln | Asn, His |
| Glu | Asp |
| His | Asn, Gln |
| Ile | Leu, Val |
| Leu | Ile, Val |
| Lys | Arg |
| Met | Leu, Ile |
| Phe | Tyr, Trp, His |
| Ser | Thr, Asn |
| Thr | Ser, Asn, Gln |
| Trp | Tyr, Phe, His |
| Tyr | Trp, Phe, His |
| Val | Ile, Leu |

Conservatively substituted analogues of the invention may have, for example, up to 10 conservative substitutions, or in another embodiment up to 5, or in yet another embodiment 3 or fewer.

### N-Acylated Analogues

All six of the Y2-Y4 selective agonists of the invention is concerned may be acylated at their N-terminus to confer resistance to other aminopeptidases. For example, acylation may be with a carbon chain having from 2 to 24 carbon atoms, and N-terminal acetylation is a particular example.

Analogues with Covalently Bound Functional Motifs

Various modifications may be made to the six Y2-Y4 selective agonists of the invention, for the purpose of improving their pharmacokinetics, pharmacodynamics and metabolic properties. Such modifications may involve linking the agonist to functional groupings (also known as motifs) known per se in the art of peptidic or proteinaceous pharmaceuticals. Three particular modifications of particular benefit in the case of the agonists with which the invention is concerned, are linkage with serum albumin binding motifs, or glycosaminoglycan (GAG) binding motifs, or PEGylation.

Serum-Albumin Binding Motifs

Serum albumin binding motifs are typically lipophilic groups, incorporated to enable a prolonged residence in the body upon administration or for other reasons, which may be coupled in various known ways to peptidic or proteinaceous molecules, for example i) via a covalent linkage to e.g. a functional group present on a side-chain amino acid residue, ii) via a functional group inserted in the peptide or in a suitable derivatized peptide, iii) as an integrated part of the peptide. For example, WO 96/29344 (Novo Nordisk A/S) and P. Kurtzhals et al. 1995 Biochemical J. 312: 725-31, describe a number of suitable lipophilic modifications which can be employed in the case of the agonists with which this invention is concerned.

Suitable lipophilic groups include optionally substituted, saturated or unsaturated, straight or branched hydrocarbon groups of from 10 to 24 carbon atoms. Such groups may form, or may form part of, a side chain to the backbone of the agonist, for example by ether, thioether, amino, ester or amide linkage to a side chain of an amino acid residue in the backbone, or to a backbone carbon or a branch from a backbone carbon of a non-peptidic linker radical in the backbone of the agonist. The chemistry strategy for attachment of the lipophilic group is not critical, but the following side chains including lipophilic groups are examples which can be linked to a backbone carbon of the agonist, or suitable branch therefrom:
CH₃(CH₂)ₙCH(COOH)NH—CO(CH₂)₂CONH— wherein n is an integer from 9 to 15,
CH₃(CH₂)ᵣCO—NHCH(COOH)(CH₂)₂CONH— wherein r is an integer form 9 to 15,
CH₃(CH₂)ₛCO—NHCH((CH₂)₂COOH)CONH— wherein s is an integer from 9 to 15,
CH₃(CH₂)ₘCONH—, wherein m is an integer from 8 to 18,
—NHCOCH((CH₂)₂COOH)NH—CO(CH₂)ₚCH₃, wherein p is an integer from 10 to 16,
—NHCO(CH₂)₂CH(COOH)NH—CO(CH₂)_{q}CH₃, wherein q is an integer from 10 to 16,
CH₃(CH₂)ₙCH(COOH)NHCO—, wherein n is an integer from 9 to 15,
CH₃(CH₂)ₚNHCO—, wherein p is an integer from 10 to 18,
—CONHCH(COOH)(CH₂)₄NH—CO(CH₂)ₘCH₃, wherein m is an integer from 8 to 18,
—CONHCH(COOH)(CH₂)₄NH—COCH((CH₂)₂COOH)NH—CO(CH₂)ₚCH₃, wherein p is an integer from 10 to 16,
—CONHCH(COOH)(CH₂)₄NH—CO(CH₂)₂CH(COOH)NH—CO(CH₂)_{q}CH₃, wherein q is an integer from 10 to 16, and
a partly or completely hydrogenated cyclopentanophenanthrene skeleton.

In one chemical synthetic strategy the lipophilic group-containing side chain is a C₁₂, C₁₄, C₁₆ or C₁₈ acyl group, for example a tetradecanoyl group, acylating an amino group present in the side chain of a residue of the backbone of the agonist.

As stated, the modification of agonists for use in accordance to provide improved serum binding characteristics is a strategy which may be applied in general, and particularly in the case of the specific agonists listed above. Thus suitable modified agonists include N—(N′-tetradecanoyl)-gammagluatamoyl-Lys13- and N—(N′-hexadecanoyl)-gammagluatamoyl-Lys13-analogues.

GAG Binding

As in the case of lipophilic serum binding motifs discussed above, the Y2-Y4 selective agonists of the invention may be modified by incorporation of the GAG binding motif as, or as part of, a side chain to the backbone of the agonist. Known GAG-binding motifs for incorporation in this way include the amino acid sequences XBBXBX and/or XBBBXXBX, wherein B is a basic amino acid residue and X is any amino acid residue. A plurality, for example three, of such sequences may be incorporated in a concatameric (straight chain) or dendrimeric (branched chain) fashion. Specific concatameric GAG motifs include Ala-Arg-Arg-Arg-Ala-Ala-Arg-Ala-Ala-Arg-Arg-Arg-Ala-Ala-Arg-Ala, and Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala (both of which may, for example be coupled through an amide bond formed between the C-terminus of the concatameric GAG-binding motif and an amino group in the side chain of a backbone amino acid of the agonist, such as the epsilon amino group of Lys13 in the agonist.

Instead of being attached to the agonist as, or as part of a side chain to a backbone residue, the GAG motif may be covalently linked to the C- or (preferably) N-terminus of the agonist, either directly or via a linker radical. Here also the GAG-binding motif may comprise the amino acid sequence XBBXBX and/or XBBBXXBX, wherein B is a basic amino acid residue and X is any amino acid residue, for example the sequence [XBBBXXBX]ₙ where n is 1 to 5, B is a basic amino acid residue and X is any amino acid residue. Such concatameric repeats tend to form alpha helices when they bind to GAG's, and consequently when fused to the C-terminal hexapeptide/last alpha helical turn, can stabilise that turn and thereby present the combined structure in an optimal way for Y4 receptor recognition. Specific examples of agonists of this type are [XBBBXXBX-XBBBXXBX]PP or [XBBBXXBX-XBBBXXBX-XBBBXXBX]PP, wherein B is a basic amino acid residue and X is any amino acid residue, particularly Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala-[Ala30]PP2-36.

The Y2-Y4 selective agonists of the invention are useful, inter alia, in indications for which prolonged exposure is desirable. For such indications in particular, the agonists preferably comprise a glycosamino glycan (GAG) binding motif as discussed above. Such motifs ensure that the agonists bind to GAGs in the extracellular matrix, and thereby ensures prolonged local exposure of the Y2 and Y4 receptors in that tissue.

Growth factors, chemokines etc bind to GAGs through patches of basic amino acids, which interact with the acidic sugars of the GAGs. These positively charged epitopes on the growth factors are usually composed of side chains from basic residues, which are not necessarily located consecutively in sequence but are often presented in close proximity by a secondary structural element such as an a-helix or a turn or by the overall three dimensional structure of the protein. Certain GAG-binding, linear sequences, discussed above, have been described, for example XBBXBX and XBBBXXBX where B represents a basic residue (Hileman et al. Bioassays 1998, 20: 156-67). These segments have been shown by circular dichroism to form α-helices upon binding to GAGs. If such sequences are placed for example in a concatameric or dendrimeric construct where for example three such sequences are presented—for example each as a ARRRAARA sequence—the resulting 24-mer peptide—for example ARRRAARA ARRRAARA-ARRRAARA—ensures a retention in the extracellular matrix similar to high molecular weight polylysine, i.e. it is not washed out during a 4 hour perfusion period (Sakharov et al. FEBS Lett 2003, 27: 6-10).

Thus Growth factors and chemokines are naturally constructed with two types of binding motifs: one binding motif for the receptor through which signal transduction is achieved and one binding motif for GAG's through which attachment and long-lasting local activity is achieved. Peptides such as PYY and NPY are neuropeptides and hormones, which are rather rapidly washed out of the tissue and are not optimized for long-lasting local activity. By attaching a GAG-binding motif to a Y2-Y4 selective agonist according to the present invention—a bi-functional molecule similar to the growth factors and chemokines is constructed having both a receptor binding epitope in the PP-fold peptide part and a GAG-binding motif. Examples include the [N-{(Ala-Arg-Arg-Arg-Ala-Ala-Ala-Arg-Ala)3}-Lys13-analogues of Y2-Y4 selective agonists of the invention

### PEGylation

In PEGylation, a polyalkyleneoxide radical or radicals, is/are covalently coupled to peptidic or proteinaceous drugs to improve effective half life in the body following administration. The term derives from the preferred polyalkyleneoxide used in such processes, namely that derived from ethylene glycol—polyethyleneglycol, or “PEG”. A suitable PEG radical may be attached to the agonist by any convenient chemistry, for example via a backbone amino acid residue of the agonist. For instance, for a molecule like e.g. PEG, a frequently used attachment group is the epsilon-amino group of lysine or the N-terminal amino group. Other attachment groups include a free carboxylic acid group (e.g. that of the C-terminal amino acid residue or of an aspartic acid or glutamic acid residue), suitably activated carbonyl groups, mercapto groups (e.g. that of a cysteine residue), aromatic acid residues (e.g. Phe, Tyr, Trp), hydroxy groups (e.g. that of Ser, Thr or OH-Lys), guanidine (e.g. Arg), imidazole (e.g. H is), and oxidized carbohydrate moieties.

When the agonist is PEGylated it usually comprises from 1 to 5 polyethylene glycol (PEG) molecules such as, e.g. 1, 2 or 3 PEG molecules. Each PEG molecule may have a molecular weight of from about 5 kDa (kiloDalton) to about 100 kDa, such as a molecular weight of from about 10 kDa to about 40 kDa, e.g., about 12 kDa or preferably no more than about 20 kDa. In a particular embodiment of the invention, PEG 40 kDa (otherwise designated PEG40000) is the PEGylating agent.

Suitable PEG molecules are available from Shearwater Polymers, Inc. and Enzon, Inc. and may be selected from SS-PEG, NPC-PEG, aldehyde-PEG, mPEG-SPA, mPEG-SCM, mPEG-BTC, SC-PEG, tresylated mPEG (U.S. Pat. No. 5,880,255), or oxycarbonyl-oxy-N-dicarboxylmideo-PEG (U.S. Pat. No. 5,122,614).

Particular examples of PEGylated agonists are [N-PEG5000-Lys13]-, [N-PEG20000Lys13]-, and [N-PEG40000Lys13]-analogues of the Y2-Y4 selective agonists of the invention

### Serum Albumin, GAG and PEG

Whether the modification to the agonist is attachment of a group to facilitate serum binding, GAG binding or improved stability via PEGylation, the serum albumin binding motif or GAG binding motif, or PEG radical may be, or may form part of, a side chain of a backbone carbon of the agonist corresponding to any of the following positions 1, 3, 6, 7, 10, 11, 12, 13, 15, 16, 18, 19, 21, 22, 23, 25, 26, 28, 29, and 32, although in the case of peptide (5) position 10 is not available.

### Conjugation to Larger Biomolecules

The Y2-Y4 selective agonists of the invention may be used as fusion proteins where they are linked for example to albumin or another protein or carrier molecule which provides beneficial pharmacokinetic or other types of properties such as for example decreased renal elimination. There are multiple chemical modifications and linkers which can be used for such a covalent attachment as known in the art, just as there are multiple proteins or carriers which can be used. Especially covalent attachment of the selective Y2-Y4 peptide agonist to albumin is preferred and at one of the positions in the PP-fold structure, which have been pointed out elsewhere herein in relation to modifications with the various motifs. Such fusion proteins can be produced through various semi-synthetic techniques where the peptide may be made through peptide synthesis as described herein and the biomolecule through recombinant technology. The fusion protein may also be made enteriely as a recombinant molecule expressed for example as a precursor molecule extended by a Gly-Lys-Arg sequence, which when expressed as a secretory protein in eukaryotic cells will be cleaved by biosynthetic enzymes and the Gly turned into the carboxyamide on the C-terminal Tyr residue of the C-terminal Y4 receptor recognition sequence.

### Helix Inducing Peptides

Acylation of the N-terminus of the agonists with which the invention is concerned has been mentioned as a means of stabilising the agonist against the action of aminopeptidases. Another stabilising modification involves the covalent attachment of a stabilizing peptide sequence of 4-20 amino acid residues covalently at the N- and/or the C-terminus, preferably the N-terminus. The amino acid residues in such a peptide are selected from the group consisting of Ala, Leu, Ser, Thr, Tyr, Asn, Gln, Asp, Glu, Lys, Arg, H is, Met and the like. In an interesting embodiment the N-terminal peptide attachment comprises 4, 5 or 6 Lys residues, for example Lys-Lys-Lys-Lys-Lys-Lys-[Ala30]PP2-36 These can be linked at the N-terminus of the PP-fold peptide agonist. A general description of such stabilizing peptide extensions is given in WO 99/46283 (Zealand Pharmaceuticals), which is hereby incorporated by reference.

The receptor agonists with which the invention is concerned may be prepared by well-known methods such as, e.g., a synthetic, semisynthetic and/or recombinant method. The methods include standard peptide preparation techniques such as, e.g., solution synthesis, and solid-phase synthesis. Based on textbook and general knowledge within the field, a person skilled in the art knows how to proceed in order to obtain the agonists and derivatives or modifications thereof.

### Clinical Indications

The Y2/Y4-specific agonists with which the invention is concerned are of value in the treatment of conditions responsive to activation of Y2 and/or Y4 receptors. Such conditions include those for which regulation of energy intake or energy metabolism, control of intestinal secretion or induction of angiogenesis, is indicated. For any such use, the agonist may be one which comprises a modification or motif which confers stability towards peptidases, serum protein binding properties, or PEGylation to prolong serum and/or tissue half-life. Especially for induction of angiogenesis, the agonist may comprise a GAG-binding motif to prolong tissue half-life and Y receptor exposure.

Diseases or conditions in which regulation of energy intake or energy metabolism is indicated include obesity and overweight, and conditions in which obesity and overweight are considered contributory factors, such as bulimia, bulimia nervosa, Syndrome X (metabolic syndrome), diabetes, type 2 diabetes mellitus or Non Insulin Dependent Diabetes Mellitus (NIDDM), hyperglycemia, insulin resistance, impaired glucose tolerance, cardiovascular disease, hypertension, atherosclerosis, coronary artery disease, myocardial infarction, peripheral vascular disease stroke, thromboembolic diseases, hypercholesterolemia, hyperlipidemia, gallbladder disease, osteoarthritis, sleep apnea, reproductive disorders such as polycystic ovarian syndrome, or cancer of the breast, prostate, or colon.

Diseases or conditions in which regulation of intestinal secretion is indicated include various forms of diarrhoea or patients suffering from hyper-secretion from their intestinal stomia.

Diseases or conditions where induction of angiogenesis is indicated include peripheral vascular disease, coronary vascular disease, myocardial infarction, stroke, conditions in which any of the foregoing is considered a contributory factor, wound healing and tissue repair.

### 1. Obesity and Overweight

PYY3-36 has been shown to decrease appetite, food intake and body weight in various rodents when administered peripherally (Batterham et al. Nature 2002, 418: 595-7; Challis et al. BBRC November 2003, 311: 915-9) as well as to decrease appetite and food intake in man also when administered peripherally (Batterham et al 2002). The animal data including studies in receptor knock out animals strongly indicate that this effect of PYY3-36 is mediated through Y2 receptors and through NPY/AgRP and POMC neurones in the arcuate nucleus. PYY levels and the PYY food responses have often been reported to be lower in obese subjects and correlates inversely with their BMI. Importantly, obese subject are not resistant to the effect of PYY as infusion of PYY3-36 for 90 minutes decreases food intake in obese subjects in a similar long lasting fashion (Batterham et al. 2003, NEJM 349: 941-48).

Much evidence from recent rodent studies has accumulated showing that PP is a powerful and efficient anorexigenic peptide when administered peripherally (Asakawa et al. Peptides 1999, 20; 1445-8; Katsuura et al. Peptides 2002, 23: 323-9; Asakawa et al. Gastroenterology 2003, 124: 1325-36). Since PP has no effect on appetite, food intake etc. in Y4 knock out animals it is very likely that PP acts through the Y4 receptor to reduce appetite and food intake (Batterham et al. 2004 Abstract S3.3 from International NPY symposium in Coimbra Portugal). PP also had effect on food intake in diet induced obese animals. pp receptors have been found especially in the brain stem in vagal motor neurones and in the nucleus tractus solitarius (nts) both of which are areas where the blood brain barrier is not efficient and where circulating hormones such as PP can get access to the neurones. Thus it is very likely that the Y4 receptors in the NTS in the brain stem are a major target through which PP acts to suppress appetite and food intake. However, recent evidence also points to the possibility that PP may also act through Y receptors in the arcuate nucleus conceivably on the POMC and perhaps also the NPY/AgRP neurones (Batterham et al. Coimbra NPY meeting abstract s3.3). Low levels of PP are found in obese subjects especially Prader-Willi syndrome (Zipf et al. J.C.E.M. 1981, 52: 1264-6, Holst et al 1983, Int. J. Obes. 7: 529-38, Glaser et al Horm. Metab. 1988, 20: 288-92) and high PP levels are found in patients with anorexia nervosa. importantly, infusion of PP in man decreases appetite and food intake for up to 24 hours (Batterham et al. JCEM 2003, 88: 3989-92). Thus, the effect of PP on food intake was observed after the PP levels in the circulation had returned to normal levels. Such long lasting effects on appetite etc, is well know also from ICV injection of especially AgRP. Importantly infusion of PP has also been shown to decrease food intake in morbidly obese patients with Prader Willi syndrome (Berntson et al 1993 Peptides 14: 497-503).

Due to the fact that PYY acting through the Y2 receptors conceivably mainly in the arcuate nucleus—inhibiting the stimulatory NPY/AgRP neurones—and PP acting through Y4 receptors mainly in the area postreama and NTS in the brain stem but also in the arcuate nucleus—but apparently stimulating the inhibitory POMC neurones (Batterham et al 2004 International NPY symposium, Coimbra abstract S3.3), the combined effect of a Y2 agonist and a Y4 agonist will have an additive or even a synergistic effect, i.e. that a more efficient effect is achieved from a combined treatment than from each of the two treatments by them selves.

PYY itself is know to be extremely emetic when administered peripherally, in fact PYY was discovered—for “the second time” —in 1989 as the biologically active entity in a chromatographic fraction of an intestinal extract causing dogs to vomit (Harding and McDonald 1989 Peptides 10: 21-24). It was concluded that PYY was the most potent, circulating emetic peptide identified and that this effect was mediated through area postreama known to have a leaky blood brain barrier. It has also been reported that PYY3-36 can cause nausea when administered peripherally to human subjects (Nastech press release 29^{th} of June 2004). From a physiological point of view it appears logically that PYY—and its biologically active conversion product—which normally first is secreted in large amounts during a very large meal or when food is dumped into the lower intestine due to various surgical procedures, is able to cause emesis and vomiting to relieve the subject from a situation of overt overeating. Interestingly, it was noted in that paper that PP given in similar doses did not cause vomiting in these dogs (Harding and McDonald 1989). PP acts through Y4 receptors also located in the area postreama of the brain stem—but does not cause emesis or vomiting. Large doses of a combined Y2-Y4 agonist peptides such as [Gln34]PP may be administered to animals such as cyno monkeys reaching plasrha levels of 12-13.000 nM without observing any vomiting of the animals or evidence of GI-tract side effects. This is surprising since [Gln34]PP has a relatively high potency on the Y2 receptor for which PYY3-36 is totally selective. Thus, surprisingly the combined Y2-Y4 selective agonist does not cause emesis to the same degree as the selective Y2 agonist—PYY3-36 compound—does. Apparently, the Y4 receptor activation—conceivably in the area postreama—prevents the emetic effect of the Y2 activation form the same compound. This property of a combined Y2-Y4 selective agonist is of great benefit for the treatment of obesity and associated conditions.

Hence, the Y2/Y4 selective agonists with which the invention is concerned are suitable for use in a subject, such as a mammal including a human, in order to regulate the energy intake. Accordingly, the invention relates to methods for altering energy intake, food intake, appetite, and energy expenditure. A method is disclosed herein for reducing energy or food intake by administering to a subject a cosmetically or therapeutically effective amount of such an agonist. In one embodiment, administration of the receptor agonist results in a decrease in the amount, either the total weight or the total volume or calorie content of the food. In another embodiment, it may result in a decrease of the intake of a food component, such as a decrease in the ingestion of lipids, carbohydrates, cholesterol, or proteins. In any of the methods disclosed herein, the preferred compounds that have been discussed in details herein could be administered. In an additional embodiment, a method is disclosed herein for reducing appetite by administering a therapeutically effective amount of such an agonist. Appetite can be measured by any means known to one of skill in the art.

For example, decreased appetite can be assessed by a psychological assessment. In such an embodiment, administration of the receptor agonist results in a change in perceived hunger, satiety, and/or fullness. Hunger can be assessed by any means known to one of skill in the art. In one embodiment, hunger is assessed using psychological assays, such as by an assessment of hunger feelings and sensory perception using e.g. a questionnaire.

In a further embodiment, a method is disclosed herein for decreasing the motility of the upper GI tract as for example decreasing gastric emptying. The method includes administering a therapeutically effective amount of such an agonist thereof to the subject, thereby decreasing GI-tract motility. It is well known that compounds which decrease gastric emptying will have a beneficial effect in also decreasing food intake as the subject is feeling more full or satiated. Both PYY3-36, the prototype Y2 agonist, and PP, the prototype Y4 agonists are know to decrease gastric emptying. A combined Y2-Y4 agonist has an additive or even a synergistic effect in inhibiting upper GI-tract motility.

In a further embodiment, a method is disclosed herein for altering energy metabolism in a subject. The method includes administering a therapeutically effective amount of such an agonist thereof to the subject, thereby altering energy expenditure. Energy is burned in all physiological processes. The body can alter the rate of energy expenditure directly, by modulating the efficiency of those processes, or changing the number and nature of processes that are occurring. For example, during digestion the body expends energy moving food through the bowel, and digesting food, and within cells, the efficiency of cellular metabolism can be altered to produce more or less heat. In a further embodiment a method is disclosed herein for any and all manipulations of the arcuate circuitry described in this application, which alter food intake coordinately and reciprocally alter energy expenditure. Energy expenditure is a result of cellular metabolism, protein synthesis, metabolic rate, and calorie utilization. Thus, in this embodiment, peripheral administration results in increased energy expenditure, and decreased efficiency of calorie utilization. In one embodiment, a therapeutically effective amount of a receptor agonist according to the invention is administered to a subject, thereby increasing energy expenditure.

In several embodiments both relating to the therapeutic use and to the cosmetic use, a Y2/Y4 selective agonist can be used for weight control and treatment, reduction or prevention of obesity, in particular any one or more of the following: preventing and reducing weight gain; inducing and promoting weight loss; and reducing obesity as measured by the Body Mass Index. As mentioned above, the invention also relates to the use of a Y2/Y4 selective agonist for controlling any one or more of appetite, satiety and hunger, in particular any one or more of the following: reducing, suppressing and inhibiting appetite; inducing, increasing, enhancing and promoting satiety and sensations of satiety; and reducing, inhibiting and suppressing hunger and sensations of hunger. The disclosure further relates to the use of a Y2/Y4 selective agonist in maintaining any one or more of a desired body weight, a desired Body Mass Index, a desired appearance and good health.

In a further or alternative aspect, the invention relates to a method for the treatment and/or prevention of reduced energy metabolism, feeding disorders, appetite disorders, overweight, obesity, bulimia, bulimia nervosa, Syndrome X (metabolic syndrome), or complications or risks associated thereto including diabetes, type 2 diabetes mellitus or Non Insulin Dependent Diabetes Mellitus (NIDDM), hyperglycemia, insulin resistance, impaired glucose tolerance, cardiovascular disease, hypertension, atherosclerosis, congestive heart failure, stroke, myocardial infarct, thromboembolic diseases, hypercholesterolemia, hyperlipidemia, gallbladder disease, osteoarthritis, sleep apnea, reproductive disorders such as polycystic ovarian syndrome, cancers of the breast, prostate, and colon, the method comprising administering to a subject such as a mammal including a human, an effective dose of one or more of a Y2/Y4 selective agonists as described herein.

### 2. Intestinal Hypersecretion

Both NPY and PYY are known to have anti-secretory effects on both the small and large intestine. Through studies on isolated human colonic tissue it was demonstrated that this effect is mediated through both Y1 and Y2 receptors and by use of TTX it was shown that a major part of the Y2 component was mediated through a neuronal component (Cox & Tough 2001 Br. J. Pharmacol. 135: 1505-12). PP also has a strong anti-secretory effect and this appears to be mediated through the Y4 receptors located on the epithelial cells and not through a neuronal mechanism (Cox & Tough 2001). Thus, due to the similar effect but mediated through different mechanisms, a combined Y2-Y4 agonist will have an additive or even a synergistic anti-secretory effect on the GI-tract. It has been shown in vivo that peripheral administration of PYY can cause a long-lasting reduction in intestinal secretion induced by vasoactive intestinal polypeptide in human subjects with ileostomies (Playford et al 1990 Lancet 335: 1555-57). It was concluded that PYY could be a therapeutic agent against diarrhoea, however, for example the natriuretic and hypertensive effects of the combined Y1 and Y2 agonist effects of the peptide has prevented this. The combined selective Y2-Y4 agonists of the present invention are particularly useful for the treatment or protection against hyper secretion of the GI-tract including various forms of diarrhoea whether or not they directly are caused by hyper-secretion. One particularly interesting indication is the hyper-secretion observed in patients with ileostomia, who often are losing large amounts of fluid.

### 3. Therapeutic Angiogenesis

A number of in vitro studies on effects on growth of vascular smooth muscle cells, hyperthrophy of ventricular cardiomyocytes as well as endothelial cell proliferation and migration have suggested that NPY may act as an angiogenic factor (Zukowska-Grojec et al. 1998 Circ. Res. 83: 187-95). Importantly, in vivo studie, s using both the mouse corneal micropocket model as well as the chick chorioallantoic membrane (CAM) assay has confirmed that NPY is a potent angiogenic factor which gives rise to vascular tree-like structures showing vasodilation as observed otherwise only with fibroblast growth factor-2 (FGF-2) and not for example vascular endothelial growth factor (VEGF) angiogenic structures (Ekstrand et al. 2003 PNAS 100: 6033-38). In the developing chick embryo NPY induced vascular sprouting from preexisting blood vessels. The effect of NPY was not observed in Y2 receptor knock out animals indicating that the Y2 receptor is responsible for the angiogenic effect of NPY (Ekstrand et al 2003). This notion is also supported by observations that the Y2 receptor is highly upregulated in ischemic vessels and the enzyme which generates the endogenous, selective Y2 ligand PYY3-36, dipentidylpeptidase-IV is also highly upregulated. The peptides of the present invention are all Y2 agonist having at least the additional property of also being Y4 agonists and being selective in respect of being poor Y1 agonists. Thus, the Y2 agonist property of these peptides makes them useful as therapeutic angiogenic agents and the Y4 agonism is beneficial in reducing or eliminating the unwanted emetic or nausea promoting effect known to be associated with high plasma levels of Y2 agonists.

In various cardiovascular diseases such as atherosclerosis for example in peripheral vessels as well as in coronary vessels is it contemplated that induction or angiogenesis would be beneficial. Also induction of angiogenesis is believed to be beneficial for securing reperfusion, after myocardial infarction. Especially FGF-2 has been proposed to be an efficient agent for induction of angiogenesis in patients with cardiovascular diseases. However, like most other angiogenic factors FGF-2 is a growth factor and has the potential of stimulating tumor growth also by providing angiogenesis. As presented above, NPY acting through Y2 receptors induces neovascularization of a similar type as induced by FGF-2, however NPY is a neuropeptide and not a classical growth factor and has not been implicated in inducing tumor growth. Thus, a Y2 agonist is a useful agent for therapeutic angiogenesis. However, it is particularly important for this use that the agonist does not show Y1 receptor agonism because this will give unwanted cardiovascular effects. This means the peptides with which the invention is concerned, which are e Y2 selective receptor agonists or which are especially useful therapeutic agents also in respect of inducing angiogenesis. They are particularly useful when modified to attach a GAG binding motif, as discussed above. To elaborate further: The action of FGF-2 as that of most other classical growth factors is partly mediated or controlled through their binding to glycosaminoglycans (GAG) in the extracellular matrix. This binding to GAGs secures that the angiogenic factor acts in an appropriate spatial and temporal fashion and that it is not washed out of the tissue rapidly. For the use of small peptides and peptide mimics such as the ones described in the present invention in therapeutic angiogenesis this is particularly important. Thus, in a preferred embodiment of the invention the peptides incorporate one or more GAG binding motif, which secures that they attach to GAGs in the extracellular matrix in order to induce optimal angiogenesis after administration. This can for example be by intravenous or intraarterial administration or for example direct administration into the coronary arteries in order to induce cardiac angiogenesis during coronary artery disease and/or post acute myocardial infarction. Similarly such a compound can be administered through intra arterial injection in the femoral artery for treatment of peripheral vascular disease. It can also be for example topical local administration to skin lesions in order to promote improved wound healing. A prolonged Y receptor exposure efficient in inducing angiogenesis can also be obtained by using a peptide according to the present invention modified with a serum albumin binding motif.

Thus in a preferred embodiment of the invention the Y2/Y4 selective agonists comprise a GAG-binding motif, which is placed in a position where it does not impair the stability of the peptide or impair the potency and selectivity of the peptide. Accordingly, in one embodiment the invention relates to the use a Y2/Y4 selective receptor agonist modifying disturbances in the angiogenesis system, especially for inducing angiogenesis such as angiogenesis associated with diseases or conditions such as e.g., cardiovascular diseases including peripheral vascular disease with symptoms such as cladicatio intermittens, coronary artery disease and myocardial infarction; tissue repair processes including wound healing in the skin, inflammatory conditions including inflammatory conditions in the gastrointestinal tract such as, e.g., ulcers, colitis, inflammatory bowel disease, Crohns disease etc.

A specific embodiment is to use the receptor agonist for inducing angiogenesis in a heart or in a blood vessel, or in a tissue such as a mucosal tissue including the gastro-intestinal mucosa and the skin.

### 3. Wound Healing

In animals where the Y2 receptor has been selectively eliminated through the deletion of its gene it has been reported that wound healing is impaired and that the associated neo-vascularization is impaired (Ekstrand et al. 2003 PNAS100: 6033-38). Thus the selective Y2-Y4 agonists of the present invention are useful to improve wound healing through their Y2 agonist property. The peptides can for this indication be administered in various ways including parenteral administration. However, a preferred route of administration is topical application e.g. in the form of a solution, dispersion, powders, sticks, creme, ointment, lotion, gel, hydrogel, transdermal delivery system including patches and plasters, etc. For topical administration they can be used as such. However, in a preferred embodiment of the invention, the peptides have been modified with one or more of the GAG-binding motifs described herein to ensure a long lasting, local effect of the peptide through binding to GAGs in the tissue.

### 4. Inflammatory Bowel Disease

PYY has previously been described for the prevention and/or treatment of inflammatory bowel disease; see WO 03/105763 to Amylin Pharmaceuticals, Inc, which is hereby incorporated by reference. Therefore the agonists with which the invention is concerned are effective in the treatment or prevention of inflammatory bowel disease as well. Accordingly, the present invention also relates to the use of the agonists described herein for such medical use. In an interesting embodiment, the peptides comprise one or more GAG-binding motifs, cf. above.

### Additional Comments Concerning Administration of Y2/Y4 Agonists for the Treatment or Prevention of Obesity and Related Diseases

During a meal a large repertoire of gastrointestinal hormones and neurotransmitter systems are activated in a carefully concerted, sequential and overlapping manner. Moreover, food components influence not only the secretion of GI hormones and the activity of various afferent neuronal pathways but these food components after absorption also influence various hormones and centers in the CNS directly. Thus the regulation of food intake and energy expenditure is a highly complex and multifaceted process. In view of this it is surprising that certain hormones such as Y2 agonists can in fact substantially affect the system when administered in a way which results in, for example only 3-4 times the plasma levels which are achieved during a meal.

Part of the problem is that administrations of such compounds—combined Y2-Y4 agonists—will have optimal effect if the compounds are given in the fasting state in an effective dose as described. If the Y2-Y4 agonists are given in a situation where the various hormonal and neuronal systems are active due to the presence of food components in the GI tract or the expectation of a meal, the effect is not seen or a much smaller effect is observed. Thus, in a preferred embodiment of the invention the combined Y2-Y4 agonist is administered in the fasting state in an effective dose either subcutaneously, nasally or through other means as described elsewhere herein. In the present context, the term “fasted state” means that the subject has not eaten any food or drink within at least the last 2 hours before administration of the combined Y2-Y4 receptor agonist such as, e.g., within at least the last 3 hours, within at least the last 4 hours, within at least the last 5 hours, within at least the last 6 hours, within at least the last 7 hours, within at least the last 8 hours, within at least the last 9 hours, within at least the last 10 hours, within at least the last 11 hours or within at least the last 12 hours before dosing.

In a subgroup of the population, the combined Y2-Y4 agonists may not have the intended action due to genetic variations such as polymorphisms in the Y4 receptor gene. Loss of function mutations in these receptors are likely to be associated with obesity. Thus, in a preferred embodiment of the invention an analysis of the Y4 receptor gene of the subject to be treated is performed in order to probe for polymorphisms/mutations in these genes and identification of such polymorphisms. Based on such an analysis an optimal treatment of the subjects can be made. For example, only subjects with normal genotype or with polymorphisms, which do not affect the function of Y4 agonists including Y2-Y4 combined agonists, should be treated with such agonists. Another possibility is to increase the dose of the combined Y2-Y4 agonist in subjects who express an impaired receptor in order to ensure an optimal effect of the drug. In the case where the obesity of a subject is caused by an impairment in the function of the Y4 receptor it could be argued that treatment with a—for example large doses—of a combined Y2-Y4 agonist is a form of replacement therapy—provided that at least some of the relevant receptor function is still left—for example in heterozygote patients. In a situation where it is not possible or not financially suitable to perform such a genetic analysis the use of a selective, combined Y2-Y4 agonist is particularly useful as it—due to its dual effect on two different targets—will be efficacious even in the cases where one of the targets are non-function or have reduced functionality due to genetic polymorphism.

An acute test may be performed to ensure that these compounds have the intended effect in the subject to be treated before a chronic treatment is started, ensuring that only subjects who are susceptible to treatment are treated.

The selective Y2-Y4 agonists of the invention may be combined in the treatment of obesity, diabetes and related diseases with the use of various other drugs targeting appetite and energy expenditure, this includes but is not limited to drugs such as GI-tract lipase inhibitors, neurotransmitter reuptake inhibitors, cannabinoid receptors antagonists and inverse agonists, as well as other types of neurotransmitter including but not limited to 5HT receptors—and/or hormone—including but not limited to GLP-1, MC4, MC3—receptor agonist or antagonists. Due to the fact that the selective Y2-Y4 agonists are targeting a homeostatic regulatory mechanism in the communication between the GI-tract and the CNS—i.e. the Y2 and the Y4 receptors normally targeted by the satiety mediating hormone PYY from the gut and PP from the pancreas—it is particularly beneficial to combine the treatment with the combined Y2-Y4 selective agonists with the treatment with a drug targeting a central, hedonic mechanism in the regulation of appetite and energy expenditure, such as the CB1 receptors, for example being part of the reward system. Thus, the use of selective Y2-Y4 agonists in the treatment of obesity and related diseases in combination with a CB1 antagonist is a preferred embodiment of this invention.

### Dosages

The therapeutically effective amount of a Y2/Y4 receptor agonist according to the invention will be dependent on specific agonist employed, the age, weight and condition of subject being treated, the severity and type of the condition or disease being treated, the manner of administration and the strength of the composition applied. For example, a therapeutically effective amount of a Y2/Y4 receptor agonist thereof can vary from about 0.01 μg per kilogram (kg) body weight to about 1 g per kg body weight, such as about 1 μg to about 5 mg per kg body weight, or about 5 μg to about 1 mg per kg body weight. In another embodiment, the receptor agonist is administered to a subject at 0.5 to 135 picomole (pmol) per kg body weight, or about 72 μmol per kg body weight. In one specific, non-limiting example from about 5 to about 50 nmol is administered as a subcutaneous injection, such as from about 2 to about 20 nmol, or about 1.0 nmol is administered as a subcutaneous injection. The exact dose is readily determined by one skilled in the art based on the potency of the specific compound (such as the receptor agonist) utilized, the age, weight, sex and physiological condition of the subject. The dose of an agonist can be a molar equivalent of the therapeutically effective dose of PYY3-36. The amounts can be divided into one or several doses for administration daily, every second day, weekly, every two weeks, monthly or with any other suitable frequency. Normally, the administration is once or twice daily.

### Methods of Administration

The agonist can be administered by any route, including the enteral (e.g. oral administration) or parenteral route. In a specific embodiment, the parenteral route is preferred and includes intravenous, intraarticular, intraperitoneal, subcutaneous, intramuscular, intrasternal injection and infusion as well as administration by the sublingual, transdermal, topical, transmucosal including nasal route, or by inhalation such as, e.g., pulmonary inhalation. In specific embodiments, the subcutaneous and/or the nasal administration route is preferred.

The receptor agonists can be administered as such dispersed in a suitable vehicle or they can be administered in the form of a suitable pharmaceutical or cosmetic composition. Such compositions are also within the scope of the invention. In the following are described suitable pharmaceutical compositions. A person skilled in the art will know how that such composition may also be suitable for cosmetic use or he will know how to adjust the compositions to cosmetic compositions by use of suitable cosmetically acceptable excipients.

### Pharmaceutical Compositions

The receptor agonists (also denoted “compounds”) according to the invention for use in medicine or cosmetics are normally presented in the form of a pharmaceutical composition comprising the specific compound or a derivative thereof together with one or more physiologically or pharmaceutically acceptable excipients.

The compounds may be administered to an animal including a mammal such as, e.g., a human by any convenient administration route such as, e.g., the oral, buccal, nasal, ocular, pulmonary, topical, transdermal, vaginal, rectal, ocular, parenteral (including inter alia subcutaneous, intramuscular, and intravenous cf. above), route in a dose that is effective for the individual purposes. A person skilled in the art will know how to chose a suitable administration route. As mentioned above, the parenteral administration route is preferred. In a specific embodiment, the receptor agonists are administered subcutaneously and/or nasally. It is well known in the art that subcutaneous injections can be easily self-administered.

A composition suitable for a specific administration route is easily determined by a medical practitioner for each patient individually. Various pharmaceutically acceptable carriers and their formulation are described in standard formulation treatises, e.g., Remington's Pharmaceutical Sciences by E. W. Martin.

The pharmaceutical composition comprising a compound according to the invention may be in the form of a solid, semi-solid or fluid composition. For parenteral use, the composition is normally in the form of a fluid composition or in the form of a semi-solid or solid form for implantation.

Fluid compositions, which are sterile solutions or dispersions can utilized by for example intravenous, intramuscular, intrathecal, epidural, intraperitoneal or subcutaneous injection of infusion. The compounds may also be prepared as a sterile solid composition, which may be dissolved or dispersed before or at the time of administration using e.g. sterile water, saline or other appropriate sterile injectable medium.

The fluid form of the composition may be a solution, an emulsion including nano-emulsions, a suspension, a dispersion, a liposomal composition, a mixture, a spray, or a aerosol (the two latter types are especially relevant for nasal administration).

Suitable mediums for solutions or dispersions are normally based on water or pharmaceutically acceptable solvents e.g. like an oil (e.g. sesame or peanut oil) or an organic solvent like e.g. propanol or isopropanol. A composition according to the invention may comprise further pharmaceutically acceptable excipients such as, e.g., pH adjusting agents, osmotically active agents e.g. in order to adjust the isotonicity of the composition to physiologically acceptable levels, viscosity adjusting agents, suspending agents, emulsifiers, stabilizers, preservatives, antioxidants etc. A preferred medium is water.

Compositions for nasal administration may also contain suitable non-irritating vehicles such as, e.g., polyethylene glycols, glycofurol, etc. as well as absorption enhancers well known by a person skilled in the art (e.g. with reference to Remington's Pharmaceutical Science)

For parenteral administration, in one embodiment the receptor agonists can be formulated generally by mixing it at the desired degree of purity, in a unit dosage injectable form (solution, suspension, or emulsion), with a pharmaceutically acceptable excipient or carrier, i.e., one that is non-toxic to recipients at the dosages and concentrations employed and is compatible with other ingredients of the composition.

Generally, the formulations are prepared by contacting the receptor agonist uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes. Due to the amphiphatic nature of the peptides described herein suitable forms also include micellar formulations, liposomes and other types of formulations comprising one or more suitable lipids such as, e.g., phospholipids and the like.

Preferably, they are suspended in an aqueous carrier, for example, in an isotonic buffer solution at a pH of about 3.0 to about 8.0, preferably at a pH of about 3.5 to about 7.4, 3.5 to 6.0, or 3.5 to about 5. Useful buffer substances include acetate, citrate, phosphate, borate, carbonate such as, e.g., sodium citrate-citric acid and sodium phosphate-phosphoric acid, and sodium acetate/acetic acid buffers.

The compositions may also be designed to controlled or prolonged delivery of the receptor agonist after administration in order to obtain a less frequent administration regimen. Normally a dosage regimen including 1-2 daily administrations is considered suitable, but within the scope of the present invention is also included other administration regimens such as, e.g., more frequent and less frequent. In order to achieve a prolonged delivery of the receptor agonist, a suitable vehicle including e.g. lipids or oils may be employed in order to form a depot at the administration site from which the receptor agonist is slowly released into the circulatory system, or an implant may be used. Suitable compositions in this respect include liposomes and biodegradable particles into which the receptor agonist has been incorporated.

In those situations where solid compositions are required, the solid composition may be in the form of tablets such as, e.g. conventional tablets, effervescent tablets, coated tablets, melt tablets or sublingual tablets, pellets, powders, granules, granulates, particulate material, solid dispersions or solid solutions.

A semi-solid form of the composition may be a chewing gum, an ointment, a cream, a liniment, a paste, a gel or a hydrogel.

Other suitable dosages forms of the pharmaceutical compositions according to the invention may be vagitories, suppositories, plasters, patches, tablets, capsules, sachets, troches, devices etc.

The dosage form may be designed to release the compound freely or in a controlled manner e.g. with respect to tablets by suitable coatings.

The pharmaceutical composition may comprise a therapeutically effective amount of a compound according to the invention.

The content of a compound of the invention in a pharmaceutical composition of the invention is e.g. from about 0.1 to about 100% w/w of the pharmaceutical composition.

The pharmaceutical compositions may be prepared by any of the method well known to a person skilled in pharmaceutical formulation.

In pharmaceutical compositions, the compounds are normally combined with a pharmaceutical excipient, i.e. a therapeutically inert substance or carrier.

The carrier may take a wide variety of forms depending on the desired dosage form and administration route.

The pharmaceutically acceptable excipients may be e.g. fillers, binders, disintegrants, diluents, glidants, solvents, emulsifying agents, suspending agents, stabilizers, enhancers, flavours, colors, pH adjusting agents, retarding agents, wetting agents, surface active agents, preservatives, antioxidants etc. Details can be found in pharmaceutical handbooks such as, e.g., Remington's Pharmaceutical Science or Pharmaceutical Excipient Handbook.

### Synthesis

Peptide agonists of the invention may be synthesized by solid phase peptide synthesis, using either an automated peptide synthesizer, or traditional bench synthesis. The solid support can be, for example, chlorotrityl (CI) or Wang (OH) resin, both of which are readily available commercially. The active groups of those resins react readily with the carboxyl group of an N-Fmoc amino acid, thereby covalently binding it to the polymer. The resin-bound amine may be deprotected by exposure to piperidine. A second N-protected amino acid may then be coupled to the resin-amino acid. These steps are repeated until the desired sequence is obtained. At the end of the synthesis, the resin-bound protected peptide may be deprotected and cleaved from the resin with trifluoroacetic acid (TFA). Examples of reagents facilitating the coupling new amino acids to the resin-bound amino acid chain are: tetra-methyluronium hexafluorophosphate (HATU), O-(1H-benzotriazole-1-yl)-N,N,N′,N′-tetramethyluronium hexafluorophosphate (HBTU), O-(1H-benzotriazole-1-yl)-N,N,N′,N′-tetramethyluronium tetrafluoroborate (TBTU), 1H-hydroxybenzotriazole (HOBt).

Peptide synthesis by solution chemistry rather than solid phase chemistry is also feasible.

Modification of a side-chain amino or carboxyl group of an amino acid in the peptide chain, for example to introduce a GAG-binding or other motif as described above, is a simple matter of selective protection and deprotection of other reactive side-chain groups not to be involved in the reaction.

### Syntheses

Peptidic agonists of the invention may be synthesized by solid phase peptide synthesis, using either an automated peptide synthesizer, or traditional bench synthesis. The solid support can be, for example, chlorotrityl (Cl) or Wang (OH) resin, both of which are readily available commercially. The active groups of those resins react readily with the carboxyl group of an N-Fmoc amino acid, thereby covalently binding it to the polymer. The resin-bound amine may be deprotected by exposure to piperidine. A second N-protected amino acid may then be coupled to the resin-amino acid. These steps are repeated until the desired sequence is obtained. At the end of the synthesis, the resin-bound protected peptide may be deprotected and cleaved from the resin with trifluoroacetic acid (TFA). Examples of reagents facilitating the coupling new amino acids to the resin-bound amino acid chain are: tetra-methyluronium hexafluorophosphate (HATU), O-(1H-benzotriazole-1-yl)-N,N,N′,N′-tetramethyluronium hexafluorophosphate (HBTU), O-(1H-benzotriazole-1-yl)-N,N,N′,N′-tetramethyluronium tetrafluoroborate (TBTU), 1H-hydroxybenzotriazole (HOBt).

Peptide synthesis by solution chemistry rather than solid phase chemistry is also feasible.

The peptides referred to herein were made by solid phase synthesis, on PAL Peg-PS resin, (amide resin (amide resin Applied Bioscience, Warrington, UK GEN913401), using Fmoc chemistry with a 5× reagent excess. The coupling was performed by HCTU throughout, solvent DMF. Fmoc removal was performed with 20% piperidine in DMF, 10-15 minutes. However, these peptides could just as well have been synthesised by various other standard peptide synthesis methods such as tBOC chemistry and solution chemistry instead of solid state etc. The synthesis is illustrated by the following description, but the other peptides with which the invention is concerned are made by similar methods:

Synthesis of [Thr30,Ile31,Gln34]hPP(SEQ ID No: 6)
In general side group protection were standard Fmoc except for:

### Arg=Fmoc Arg(Pbf) OH

### Asn, Gin=Fmoc Asn(Trt)-OH

Thr, Ser, Asp, Glu, Tyr=tButyl
Ala-Ser 22-23=Fmoc AlaSer pseudoproline

The peptide was synthesized by solid phase synthesis, on PAL Peg-PS resin (a resin which will generate the biologically important carboxyamide group upon cleavage), using Fmoc chemistry with a 5 fold molar reagent excess. The coupling was performed by HCTU throughout using DMF as solvent. Fmoc removal after each coupling step was performed with 20% piperidine in DMF for 10-15 minutes. The coupling was checked after each step by quantitative ninhydrin (Kaiser) assay. In certain cases double couplings could be performed.

The resin can be divided into parts to produce separate batches of peptide.

The peptides are cleaved of the resin with TFA, silane and water 94:3:3. The solvent was removed by a stream of nitrogen and the residue was washed with ether and air dried, dissolved in 10% acetic acid and freeze dried.

In one example of a purification method the crude material is purified by reverse phase HPLC using ACE 300 A C18 columns, typically 250 mm×10 mm flow 2 ml/min, with 215 nm detection.

Buffer A=0.05% TFA in water
Buffer B=60% MeCN+0.05% TFA and water

A typical gradient used for the sequences included herein is a gradient of 20% to 90% buffer B over 20 mins collect the main peak. Peptide identify is confirmed by mass spectroscopy using for example MALDI TOF ionisation technique (Electrospray or Atmospheric Pressure Chemical Ionisation techniques are other examples of ionisation techniques that can be used). Purity is checked by for example analytical HPLC method A. Fractions containing the product are pooled and freeze dried to yield the trifluoroacetate salt of the peptide product.

As mentioned, the other peptides with which the invention is concerned are made according to the above method or by that method with minor variations well known in the peptide synthesis art.

In general side group protection is standard Fmoc except for:

Arg=Fmoc Arg(Pbf) OH

Asn, Gin=Fmoc Asn(Trt)-OH

Thr, Ser, Asp, Glu, Tyr=tButyl

Ala-Ser 22-23=Fmoc AlaSer pseudoproline

### Summary of Analytical Data for the Peptides:—

| | | Molecular | | Meassured | Rt | | Analytical |
|---|---|---|---|---|---|---|---|
| Sequence | Structure | formular | Mw | mass m/z | min | Purity % | method |
| 9 | [Ile31, Gln34]PP2-36 | C185 H288 | 4142 | 4143 | 9.75 | 95.1 | A |
| | | N54 O55 S2 | | | | | |
| 6 | [Thr30, Ile31, Gln34]PP | C184 H286 | 4183 | 4190 | 9.66 | 100.0 | A |
| | | N54 O56 S | | | | | |
| 7 | [Thr30, Ile31, Gln34]PP2-36 | C181 H281 | 4112 | 4115 | 9.47 | 98.0 | A |
| | | N53 O55 S | | | | | |
| 4 | [Thr30, Gln34]PP | C184 H286 | 4183 | 4182 | 13.78 | 98.0 | A |
| | | N54 O56 S | | | | | |
| 5 | [Thr30, Gln34]PP2-36 | C181 H281 | 4112 | 4111 | 9.62 | 97.9 | A |
| | | N53 O55 S | | | | | |
| 8 | [Glu10, Thr30, Ile31, Gln34]PP2-36 | C182 H283 | 4126 | 4116 | 10.77 | 100 | A |
| | | N53 O55 S | | | | | |
| 18 | [Gln34]PP2-36 | C182 H283 | 4142 | 4141 | 8.55 | 95.7 | A |
| | | N53 O54 S2 | | | | | |

### Analytical HPLC Method A

Column=Vydac C18 Peptide-Protein column, 250×4.6 mm
Buffer A=0.05% TFA in water

### Buffer B=0.05% TFA in 100% MeCN

### Gradient=0% B to 60% B in 20 min

Flow rate=1.00 mL/min

### Wavelength=215 nm

Mass spectroscopy=MALDI-TOF with gentisic acid or acyanohydroxy cinnamic acid as matrix.

### Biological Assays and Results

### A. In Vitro Assays to Determine Peptide Potency

### Human Y2 Receptor Potency Assay

Potency of the test compounds on the human Y2 receptor is determined by performing dose-response experiments in COS-7 cells transiently transfected with the human Y2 receptor as well as a promiscuous G protein, Gqi5 which ensures that the Y2 receptor couples through a Gq pathway leading to an increase in inositol phosphate turnover.

Phosphatidylinositol turnover—One day after transfection COS-7 cells are incubated for 24 hours with 5 μCi of [3H]-myo-inositol (Amersham, PT6-271) in 1 ml medium supplemented with 10% fetal calf serum, 2 mM glutamine and 0.01 mg/ml gentamicin per well. Cells are washed twice in buffer, 20 mM HEPES, pH 7.4, supplemented with 140 mM NaCl, 5 mM KCl, 1 mM MgSO₄, 1 mM CaCl2, 10 mM glucose, 0.05% (w/v) bovine serum; and are incubated in 0.5 ml buffer supplemented with 10 mM LiCl at 37 C for 30 min. After stimulation with various concentrations of peptide for 45 min at 37 C, cells are extracted with 10% ice-cold perchloric acid followed by incubation on ice for 30 min. The resulting supernatants are neutralized with KOH in HEPES buffer, and the generated [3H]-inositol phosphate are purified on Bio-Rad AG 1-X8 anion-exchange resin and counted in a beta counter. Determinations are made in duplicates. EC50 values were calculated using a standard pharmacological data handling software, Prism 3.0 (graphPad Sofware, San Diego, USA).

### Human Y4 Receptor Potency Assay

Protocol as for the Y2 potency assay, except that human Y4-transformed COS-7 cells are used.

### Human Y1 Receptor Potency Assay

Protocol as for the Y2 potency assay, except that human Y1-transformed COS-7 cells are used.

### Human Y5 Receptor Potency Assay

Protocol as for the Y2 potency assay, except that human Y5-transformed COS-7 cells are used

The results of testing the agonists of the invention, and comparison Y2-Y4 selective agonists disclosed in International Patent Application WO 2005/089790 in the above potency assays are given in Table 1:

TABLE 1

| Seq ID | | hY1 | | hY2 | | hY4 | | hY5 | |
|---|---|---|---|---|---|---|---|---|---|
| No | Name | EC50 (M) | (N) | EC50 (M) | (N) | EC50 (M) | (N) | EC50 (M) | (N) |
| 1 | hNPY1-36 | 1.7E−09 | (33) | 1.0E−09 | (17) | 1.1E−07 | (6) | 7.6E−09 | (32) |
| 2 | hPYY | 5.5E−10 | (5) | 1.6E−10 | (7) | 3.3E−08 | (5) | 8.3E−09 | (5) |
| 10 | hPYY 3-36 | 1.1E−07 | (21) | 2.5E−10 | (51) | >1E−06 | (16) | 5.7E−08 | (19) |
| 3 | hPP | 2.1E−07 | (17) | >1E−06 | (17) | 6.9E−10 | (55) | 3.4E−08 | (18) |
| 11 | [Gln34]hPP | 6.4E−07 | (29) | 2.1E−09 | (32) | 7.6E−10 | (37) | 1.6E−08 | (29) |
| 18 | Gln34]hPP2-36 | 2E−06 | (3) | 1.97E09 | (3) | 1.87E−09 | (4) | 1.94E−08 | 3) |
| 12 | [Gln34]hPP3-36 | >1E−06 | (5) | 9.2E−10 | (5) | 4.3E−09 | (5) | 3.3E−08 | (5) |
| 13 | [Val3, Gln34]hPP | 9.7E−08 | (3) | 1.3E−08 | (3) | 1.5E−09 | (3) | 2.6E−08 | (4) |
| 14 | [Ile31, Gln34]hPP | 4.7E−08 | (3) | 7.5E−09 | (3) | 1.1E−09 | (4) | 1.9E−08 | (4) |
| 9 | [Ile31, Gln34]hPP2-36 | 3.7E−07 | (4) | 3.4E−09 | (4) | 1.2E−09 | (4) | 8.1E−09 | (4) |
| 6 | [Thr30, Ile31, Gln34]hPP | >1E−06 | (6) | 9.1E−09 | (6) | 8.0E−10 | (6) | 1.7E−07 | (6) |
| 7 | [Thr30, Ile31, Gln34]hPP2-36 | >1E−06 | (6) | 4.8E−09 | (6) | 1.9E−09 | (6) | 6.7E−08 | (6) |
| 8 | [Glu10, Thr30, Ile31, Gln34]hPP2-36 | >1E−06 | (4) | 3.6E−09 | (4) | 2.7E−09 | (4) | 6.6E−08 | (4) |
| 15 | [Leu30, Gln34]hPP | 1.0E−07 | (4) | 8.5E−09 | (3) | 4.4E−09 | (3) | 5.2E−08 | (4) |
| 4 | [Thr30, Gln34]hPP | >1E−06 | (3) | 4.0E−09 | (3) | 9.2E−10 | (3) | 5.8E−07 | (3) |
| 5 | [Thr30, Gln34]hPP2-36 | >1E−06 | (4) | 3.3E−09 | (4) | 2.2E−09 | (4) | 1.3E−07 | (4) |
| 16 | [His26, Gln34]hPP | 1.2E−07 | (4) | 5.9E−09 | (3) | 9.2E−09 | (3) | 6.0E−08 | (4) |
| 17 | [Ala1, Glu4, Arg26, Met30]hPYY | 1.7E−07 | (15) | 1.8E−09 | (17) | 2.9E−09 | (15) | 4.4E−08 | (16) |

Note:
SEQ ID Nos: 11-17 are disclosed in WO 2005/089790 and are included in the above Table for comparison purposes only.
SEQ ID No: 10 is the natural circulating degradation product of hPYY.

### B. In Vivo Studies to Determine the Effect of the Peptides on Food Intake and Body Weight

### Effect of Acute Administration of Test Compounds on Mouse Food Intake

The effects of test compounds on body weight and food intake in male C57BU6J mice habituated to the daily presentation of a palatable wet mash diet were investigated. Animals were maintained on normal-phase lighting. Test compounds were administered orally, and measurements made over the following 24 hours. All experiments included vehicle-treated control groups.

### Materials and Methods

Male C57BU6J mice (weight range 20-25 g) were individually housed in polypropylene cages at a temperature of 21±4° C. and 55%±20% humidity. Animals were maintained on a normal phase light-dark cycle (lights off for 12 h from 19:00-07:00 h) during which time the room were illuminated by red light. Animals had free access to a standard pelleted rodent diet and tap water at all times. In addition, animals were habituated to a daily presentation of a wet mash diet (1 part powdered chow: 1.5 parts tap water) placed on a dish on the cage floor. Animals were accustomed to these conditions for at least ten days before experimentation.

On the day prior to testing, the experimental animals were randomly allocated to suitable treatment groups. Animals were weighed and 2 h wet mash intake were calculated (to the nearest 0.1 g). Simple in-house data and statistical analysis were performed to investigate whether there are any significant differences between the treatment groups at baseline. Animals were reallocated into different groups if necessary to resolve any significant differences and ensure that the groups were balanced before drug treatment.

On the test day, animals were briefly removed from the home cage, weighed and dosed orally with either vehicle (water), test compound (in aqueous solution), or a positive control.

Test compounds were administered 30 minutes before the presentation of ‘wet mash’. Food pellets were removed were weighed (to the nearest 0.1 g) at the time of drug administration. Wet mash was weighed 1, 2, and 4 h after presentation. The mash was replaced with a fresh quantity of wet mash at the 4 hour time point. Wet mash was re-weighed at the 6 hour time point. Wet mash was replaced with a known quantity of standard pellets at the 6 hour time point. Food pellets and animals will were weighed 24 h post dosing. Food intakes of the different groups of animals were measured concurrently.

Results (body weights (g) at 0, 24 h; change in body weight (g) over 24; food intake at 1, 2, 4, 6 and 24 h and between 1-2 h, 2-4 h, 4-6 h) were expressed as mean values±SEM. Food intake was expressed in g. Statistical comparisons between the food intakes and body weights of different groups of mice were made. P<0.05 was considered to be statistically significant.

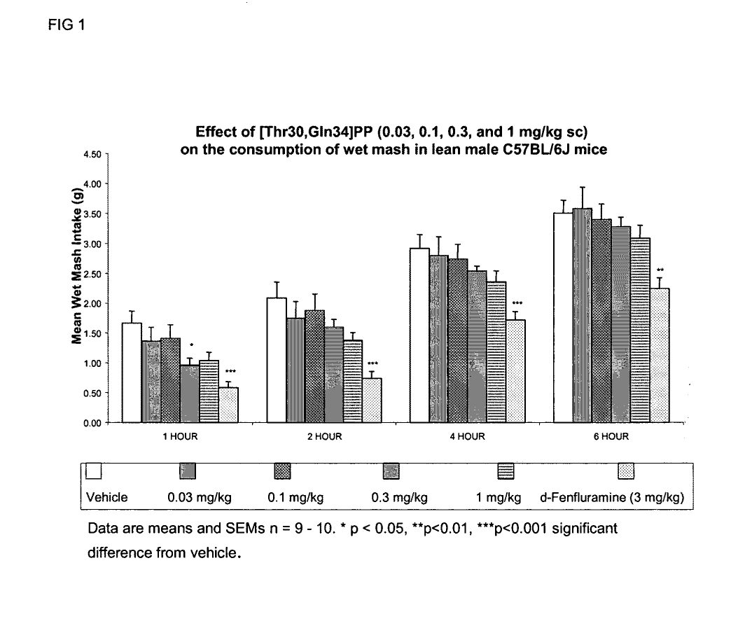

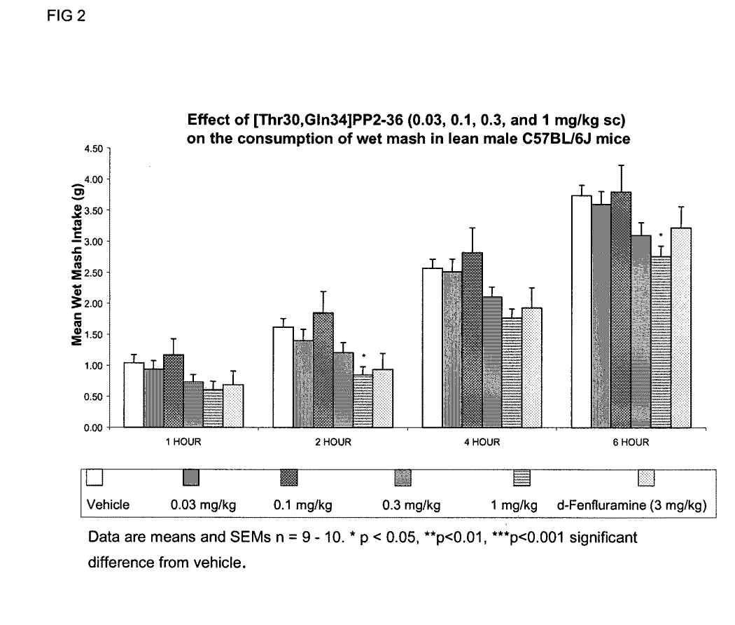

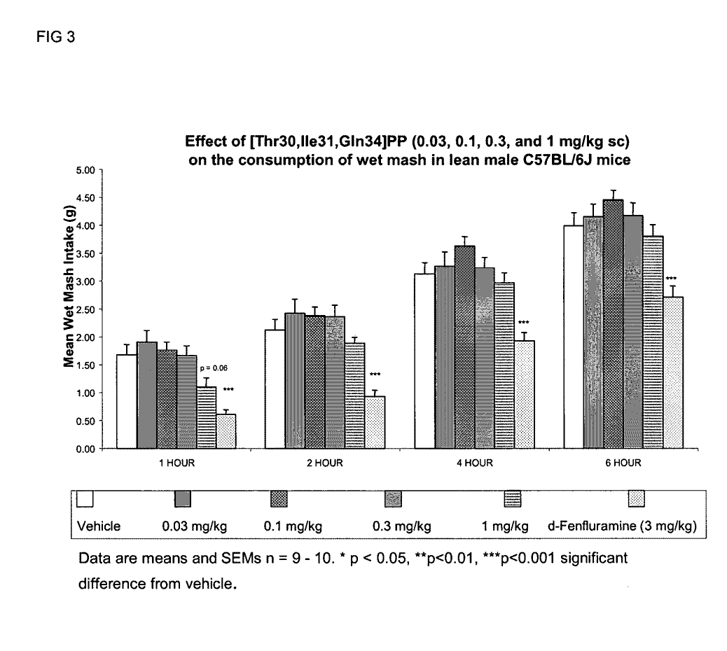

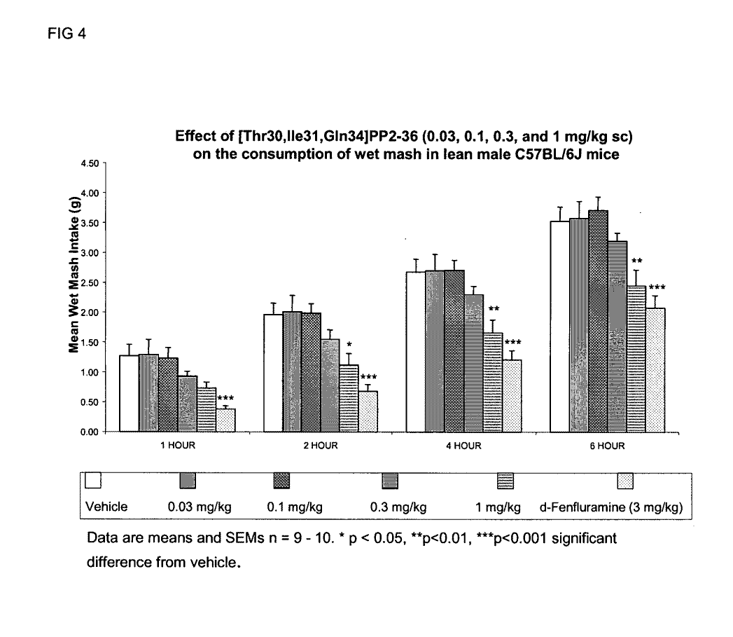

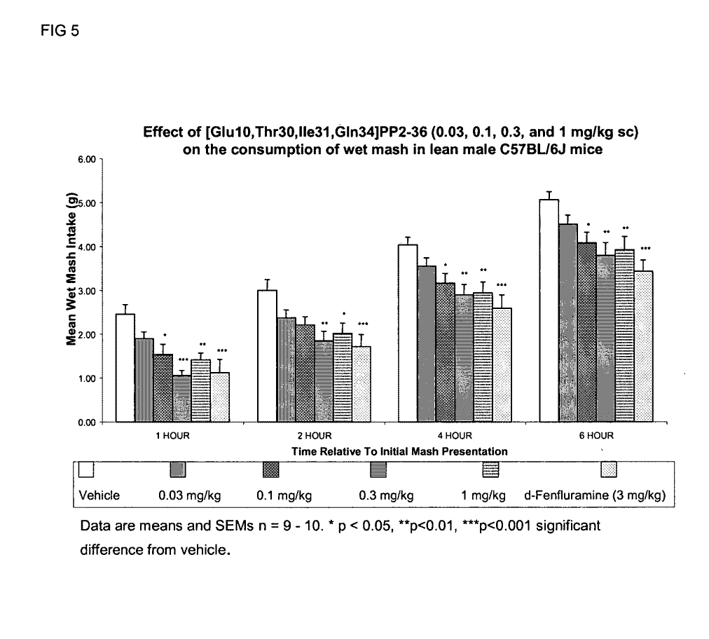

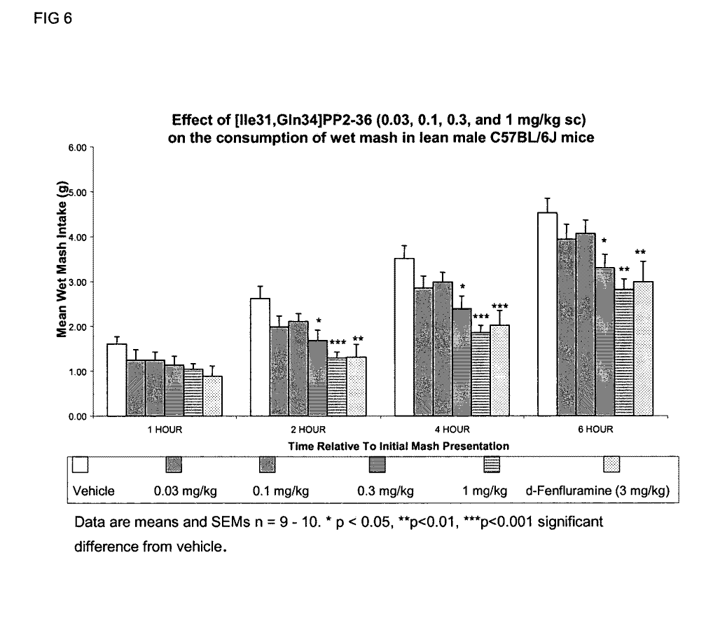

The results for each of the test peptides (1) (6) (SEQ ID Nos 4-9) of the present invention as test compounds are shown in FIGS. 1 to 6.

### Effect of Chronic Administration of Test Compounds on Body Weight and Food and Water Intake in Dietary-Induced Obese Mice

The effects of chronic administration (up to 28 days) of test compounds on body weight and daily food intake in C57BL/6J mice exhibiting obesity due to access to a high fat diet were investigated.

### Materials and Methods

Fifty seven male C57BU6J mice (4-6 weeks of age) were group-housed in polypropylene cages with free access to a high fat diet (D12451 45% of kcal derived from fat; Research Diets, New Jersey, USA) and tap water. Eight mice were group-housed in polypropylene cages with free access to a control diet (D12450B 10% of kcal derived from fat; Research Diets, New Jersey, USA) and tap water. Animals were maintained at 21±4° C. and 55±20% humidity on a normal phase 12 h light-dark cycle.

Animals were exposed to the relevant diet for 14 weeks. During this time body weight were recorded weekly. Animals were singly housed at week 14 for a 2 week period and placed on a reverse phase light-dark cycle with lights off at 10:00 and on at 18:00. At the start of the study, animals were weighed and allocated into 7 weight-matched treatment groups. Animals underwent a 14-day baseline run-in period during which time all mice were dosed subcutaneously once a day with vehicle. Towards the end of this baseline treatment animals were weighed and allocated into 6 weight-matched treatment groups. At this stage two mice were removed from the study due to poor condition and/or excessive weight loss.

Subsequently, mice were dosed subcutaneously (injection volume 3 ml/kg such that a 25 g mouse receives 0.075 ml) once daily with vehicle (physiological saline) or test compound (in solution in physiological saline). Food intake and body weight were recorded daily.

Body weights and food intake were expressed as mean values±SEM. Body weight data were analysed by ANCOVA followed by appropriate comparisons (two-tailed) to determine significant differences from the control group. P<0.05 was considered to be statistically significant. Daily food intake data were analysed by ANOVA followed by appropriate comparisons (two-tailed).

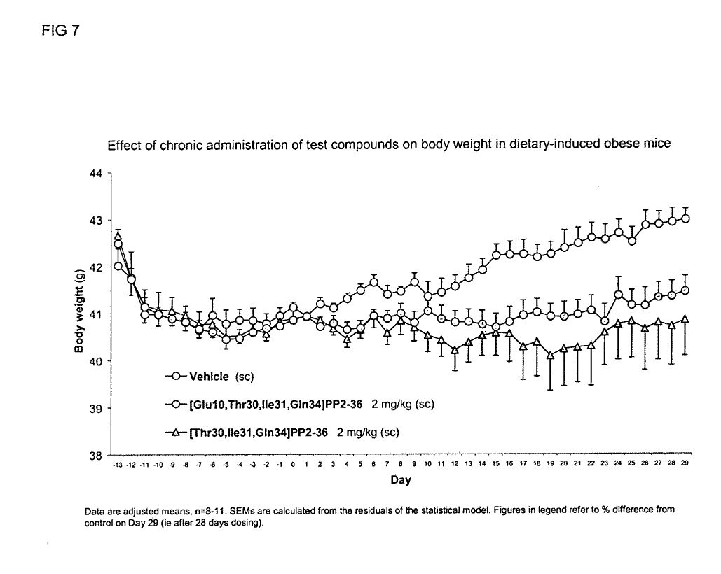

FIG. 7 shows the effect of chronic administration of two of the peptides of the invention on the body weight of dietary-induced obese mice, relative to saline control.

### Sequences:

hNPY

(SEQ ID No: 1)

H₂N-Tyr--Pro-Ser-Lys-Pro-Asp-Asn-Pro-Gly-Glu-Asp-
Ala-Pro-Ala-Glu-Asp-Met-Ala-Arg-Tyr-Tyr-Ser-Ala-
Leu-Arg-His-Tyr-Ile-Asn-Leu-Ile-Thr-Arg-Gln-Arg-
Tyr-CONH₂
hPYY

(SEQ ID No: 2)

H₂NTyr-Pro-Ile-Lys-Pro-Glu-Ala-Pro-Gly-Glu-Asp-
Ala-Ser-Pro-Glu-Glu-Leu-Asn-Arg-Tyr-Tyr-Ala-Ser-
Leu-Arg-His-Tyr-Leu-Asn-Leu-Val-Thr-Arg-Gln-Arg-
Tyr-CONH₂
hPP

(SEQ ID No: 3)

H₂N-Ala-Pro-Leu-Glu-Pro-Val-Tyr-Pro-Gly-Asp-Asn-
Ala-Thr-Pro-Glu-Gln-Met-Ala-Gln-Tyr-Ala-Ala-Asp-
Leu-Arg-Arg-Tyr-Ile-Asn-Met-Leu-Thr-Arg-Pro-Arg-
Tyr-CONH₂
[Thr30, Gln34]hPP

(SEQ ID No: 4)

H₂N-Ala-Pro-Leu-Glu-Pro-Val-Tyr-Pro-Gly-Asp-Asn-
Ala-Thr-Pro-Glu-Gln-Met-Ala-Gln-Tyr-Ala-Ala-Asp-
Leu-Arg-Arg-Tyr-Ile-Asn-Thr-Leu-Thr-Arg-Gln-Arg-
Tyr-CONH₂
[Thr30, Gln34]hPP2-36

(SEQ ID No: 5)

H₂N-Pro-Leu-Glu-Pro-Val-Tyr-Pro-Gly-Asp-Asn-Ala-
Thr-Pro-Glu-Gln-Met-Ala-Gln-Tyr-Ala-Ala-Asp-Leu-
Arg-Arg-Tyr-Ile-Asn-Thr-Leu-Thr-Arg-Gln-Arg-Tyr-
CONH₂
[Thr30, Ile31, Gln34]hPP

(SEQ ID No: 6)

H₂N-Ala-Pro-Leu-Glu-Pro-Val-Tyr-Pro-Gly-Asp-Asn-
Ala-Thr-Pro-Glu-Gln-Met-Ala-Gln-Tyr-Ala-Ala-Asp-
Leu-Arg-Arg-Tyr-Ile-Asn-Thr-Ile-Thr-Arg-Gln-Arg-
Tyr-CONH₂
[Thr30, Ile31, Gln34]hPP2-36

(SEQ ID No: 7)

H₂N-Pro-Leu-Glu-Pro-Val-Tyr-Pro-Gly-Asp-Asn-Ala-
Thr-Pro-Glu-Gln-Met-Ala-Gln-Tyr-Ala-Ala-Asp-Leu-
Arg-Arg-Tyr-Ile-Asn-Thr-Ile-Thr-Arg-Gln-Arg-Tyr-
CONH₂
[Glu10, Thr30, Ile31, Gln34]hPP2-36

(SEQ ID No: 8)

H₂N-Pro-Leu-Glu-Pro-Val-Tyr-Pro-Gly-Glu-Asn-Ala-
Thr-Pro-Glu-Gln-Met-Ala-Gln-Tyr-Ala-Ala-Asp-Leu-
Arg-Arg-Tyr-Ile-Asn-Thr-Ile-Thr-Arg-Gln-Arg-Tyr-
CONH₂
[Ile31, Gln34]hPP2-36

(SEQ ID No: 9)

H₂N-Pro-Leu-Glu-Pro-Val-Tyr-Pro-Gly-Asp-Asn-Ala-
Thr-Pro-Glu-Gln-Met-Ala-Gln-Tyr-Ala-Ala-Asp-Leu-
Arg-Arg-Tyr-Ile-Asn-Met-Ile-Thr-Arg-Gln-Arg-Tyr-
CONH₂
hPYY3-36

(SEQ ID No: 10)

H₂N-Tyr-Pro-Ile-Lys-Pro-Glu-Ala-Pro-Gly-Glu-Asp-
Ala-Ser-Pro-Glu-Glu-Leu-Asn-Arg-Tyr-Tyr-Ala-Ser-
Leu-Arg-His-Tyr-Leu-Asn-Leu-Val-Thr-Arg-Gln-Arg-
Tyr-CONH₂
[Gln34]hPP

(SEQ ID No: 11)

H₂N-Ala-Pro-Leu-Glu-Pro-Val-Tyr-Pro-Gly-Asp-Asn-
Ala-Thr-Pro-Glu-Gln-Met-Ala-Gln-Tyr-Ala-Ala-Asp-
Leu-Arg-Arg-Tyr-Ile-Asn-Met-Leu-Thr-Arg-Gln-Arg-
Tyr-CONH₂
[Gln34]hPP3-36

(SEQ ID No: 12)

H₂N-Leu-Glu-Pro-Val-Tyr-Pro-Gly-Asp-Asn-Ala-Thr-
Pro-Glu-Gln-Met-Ala-Gln-Tyr-Ala-Ala-Asp-Leu-Arg-
Arg-Tyr-Ile-Asn-Met-Leu-Thr-Arg-Gln-Arg-Tyr-CONH₂
[Val3, Gln34]hPP

(SEQ ID No: 13)

H₂N-Ala-Pro-Val-Glu-Pro-Val-Tyr-Pro-Gly-Asp-Asn-
Ala-Thr-Pro-Glu-Gln-Met-Ala-Gln-Tyr-Ala-Ala-Asp-
Leu-Arg-Arg-Tyr-Ile-Asn-Met-Leu-Thr-Arg-Gln-Arg-
Tyr-CONH₂
[Ile31, Gln34]hPP

(SEQ ID No: 14)

H₂N-Ala-Pro-Leu-Glu-Pro-Val-Tyr-Pro-Gly-Asp-Asn-
Ala-Thr-Pro-Glu-Gln-Met-Ala-Gln-Tyr-Ala-Ala-Asp-
Leu-Arg-Arg-Tyr-Ile-Asn-Met-Ile-Thr-Arg-Gln-Arg-
Tyr-CONH₂
[Ile31, Gln34]hPP

(SEQ ID No: 15)

H₂N-Ala-Pro-Leu-Glu-Pro-Val-Tyr-Pro-Gly-Asp-Asn-
Ala-Thr-Pro-Glu-Gln-Met-Ala-Gln-Tyr-Ala-Ala-Asp-
Leu-Arg-Arg-Tyr-Ile-Asn-Met-Ile-Thr-Arg-Gln-Arg-
Tyr-CONH₂
[His26, Gln34]hPP

(SEQ ID No: 16)

H₂N-Ala-Pro-Leu-Glu-Pro-Val-Tyr-Pro-Gly-Asp-Asn-
Ala-Thr-Pro-Glu-Gln-Met-Ala-Gln-Tyr-Ala-Ala-Asp-
Leu-Arg-His-Tyr-Ile-Asn-Met-Leu-Thr-Arg-Gln-Arg-
Tyr-CONH₂
[Ala1, Glu4, Arg26, Met30]hPYY

(SEQ ID No: 17)

H₂N-Ala-Pro-Ile-Glu-Pro-Glu-Ala-Pro-Gly-Glu-Asp-
Ala-Ser-Pro-Glu-Glu-Leu-Asn-Arg-Tyr-Tyr-Ala-Ser-
Leu-Arg-Arg-Tyr-Leu-Asn-Met-Val-Thr-Arg-Gln-Arg-
Tyr-CONH₂
[Gln34]hPP2-36

(SEQ ID No: 18)

H₂N-Pro-Leu-Glu-Pro-Val-Tyr-Pro-Gly-Asp-Asn-Ala-
Thr-Pro-Glu-Gln-Met-Ala-Gln-Tyr-Ala-Ala-Asp-Leu-
Arg-Arg-Tyr-Ile-Asn-Met-Leu-Thr-Arg-Gln-Arg-Tyr-
CONH₂

1. A peptide selected from the group consisting of
(1) [Thr30,Gln34]hPP (SEQ ID NO:4),
(2) [Thr30,Gln34]hPP2-36 (SEQ ID NO:5);
(3) [Thr30,Ile31,Gln34]hPP (SEQ ID NO:6);
(4) [Thr30,Ile31,Gln34]hPP2-36 (SEQ ID NO:7);
(5) [Glu10,Thr30,Ile31,Gln34]hPP2-36 (SEQ ID NO:8); and
(6) [Ile31,Gln34]hPP2-36 (SEQ ID NO:9);
(7) [Gln34]hPP2-36 (SEQ ID NO:18); and
analogues thereof which are
(a) conservatively substituted in one or more positions other than position 34 in cases (1) to (7), and position 30 in cases (1) to (5), and position 31 cases (3) to (6) and position 10 in case (5); and/or
(b) N-terminally acylated, PEGylated, or covalently coupled to a serum albumin binding motif, a glycosaminoglycan binding motif or a helix inducing motif, said covalent coupling being to a residue of the peptide or to a residue substituted in peptide which provides a functional group for such covalent binding.

2. The peptide of claim 1 which is acylated at its N-terminus.

3. The peptide of claim 2 which is acylated at its N-terminus with a carbon chain having from 2 to 24 carbon atoms, for example having an N-terminal N—(N′-tetradecanoyl)-gammaglutamoyl group.

4. The peptide of claim 2 which is acetylated at its N-terminus.

5. The peptide of claim 1 which comprises a serum albumin binding motif, or a glycosaminoglycan (GAG) binding motif, or a helix inducing motif, or is PEGylated.

6. The peptide of claim 5 comprising a serum albumin binding motif which is a lipophilic group.

7. The peptide of claim 6 wherein the lipophilic group comprises an optionally substituted, saturated or unsaturated, straight or branched hydrocarbon group of from 10 to 24 carbon atoms.

8. The peptide of claim 6, wherein the lipophilic group is, or is part of, a side chain to the backbone of the peptide.

9. The peptide of claim 8 wherein the lipophilic group-containing side chain is connected to a residue in the backbone via an ether, thioether, amino, ester or amide bond.

10. The peptide of claim 9 wherein the lipophilic group-containing side chain is selected from the group consisting of:
CH₃(CH₂)ₙCH(COOH)NH—CO(CH₂)₂CONH—, wherein n is an integer from 9 to 15,
CH₃(CH₂)ᵣCO—NHCH(COOH)(CH₂)₂CONH—, wherein r is an integer form 9 to 15,
CH₃(CH₂)ₛCO—NHCH((CH₂)₂COOH)CONH—, wherein s is an integer from 9 to 15,
CH₃(CH₂)ₘCONH—, wherein m is an integer from 8 to 18,
—NHCOCH((CH₂)₂COOH)NH—CO(CH₂)ₚCH₃, wherein p is an integer from 10 to 16,
—NHCO(CH₂)₂CH(COOH)NH—CO(CH₂)_{q}CH₃, wherein q is an integer from 10 to 16,
CH₃(CH₂)ₙCH(COOH)NHCO—, wherein n is an integer from 9 to 15,
CH₃(CH₂)ₚNHCO—, wherein p is an integer from 10 to 18,
—CONHCH(COOH)(CH₂)₄NH—CO(CH₂)ₘCH₃, wherein m is an integer from 8 to 18,
—CONHCH(COOH)(CH₂)₄NH—COCH((CH₂)₂COOH)NH—CO(CH₂)ₚCH₃, wherein p is an integer from 10 to 16,
—CONHCH(COOH)(CH₂)₄NH—CO(CH₂)₂CH(COOH)NH—CO(CH₂)_{q}CH₃, wherein q is an integer from 10 to 16, and
a partly or completely hydrogenated cyclopentanophenanthrene skeleton.

11. The peptide of claim 8 wherein the lipophilic group-containing side chain is a C₁₂, C₁₄, C₁₆ or C₁₈ acyl group acylating an amino group present in the side chain of a residue of the backbone of the peptide.

12. The peptide of claim 11 wherein the lipophilic group-containing side chain is a tetradecanoyl group acylating an amino group present in the side chain of a residue of the backbone of the peptide.

13. The peptide of claim 7 wherein the lipophilic group-containing side chain is formed by acylation of the epsilon amino group of a Lys13 substitution in the peptide.

14. The peptide of claim 5, wherein the GAG binding motif is an amino acid sequence which is, or is part of, a side chain to the backbone of the peptide.

15. The peptide of claim 14 wherein the GAG-binding motif comprises the amino acid sequence XBBXBX (SEQ ID NO:20) and/or XBBBXXBX (SEQ ID NO:21), wherein B is a basic amino acid residue and X is any amino acid residue

16. The peptide of claim 14 wherein the GAG-binding motif is concatameric or dendrimeric.

17. The peptide of claim 14 wherein the GAG-binding motif is Ala-Arg-Arg-Arg-Ala-Ala-Arg-Ala-Ala-Arg-Arg-Arg-Ala-Ala-Arg-Ala (SEQ ID NO:22) coupled through an amide bond formed between the C-terminus of the concatameric GAG-binding motif and the epsilon amino group of a Lys13 substitution in the peptide,

18. The peptide of claim 14 wherein the GAG-binding motif is Ala-Arg-Arg-Arg-Ala-Ala-Arg-Ala-Ala-Arg-Arg-Arg-Ala-Ala-Arg-Ala-Ala-Arg-Arg-Arg-Ala-Ala-Arg-Ala (SEQ ID NO:23) coupled through an amide bond formed between the C-terminus of the concatameric GAG-binding motif and the epsilon amino group of a Lys13 substitution in the peptide.

19. The peptide of claim 5 wherein the GAG binding motif is covalently linked to the C- or N-terminus of the peptide, either directly or via a linker radical.

20. The peptide of claim 19 wherein the GAG binding motif is covalently linked either directly or via a linker radical to the N-terminus of the peptide.

21. The peptide of claim 19 wherein the GAG-binding motif comprises the amino acid sequence XBBXBX (SEQ ID NO:20) and/or XBBBXXBX (SEQ ID NO:21), wherein B is a basic amino acid residue and X is any amino acid residue.

22. The peptide of claim 19 wherein, in the agonist, the GAG-binding motif comprises the amino acid sequence [XBBBXXBX (SEQ ID NO:21)]ₙ where n is 1 to 5, B is a basic amino acid residue and X is any amino acid residue.

23. The peptide of claim 15 wherein the GAG binding motif is an (Ala-Arg-Arg-Arg-Ala-Ala-Ala-Arg-Ala)₃ (SEQ ID NO:29) acylation of the epsilon amino group of a Lys13 substitution in the peptide.

24. The peptide of claim 5 wherein the PEG is a polyethylene glycol or a polyethylene oxide having a molecular weight of at the most about 20 kDa.

25. The peptide of claim 5 which is a PEG adduct on the epsilon amino group of a Lys13 substitution in the peptide

26. The peptide of claim 5 wherein the helix inducing peptide is covalently linked, either directly or via a linker radical, to the C- or N-terminus of the agonist,

27. The peptide of claim 5 wherein the helix inducing peptide is covalently linked, either directly or via a linker radical, to the N-terminus of the agonist,

28. The peptide of claim 26 wherein the helix inducing peptide has 4-20 amino acid residues selected from the group consisting of Ala, Leu, Ser, Thr, Tyr, Asn, Gln, Asp, Glu, Lys, Arg, H is, Met, Orn, and amino acid residues of formula —NH—C(R1)(R2)-CO— wherein R1 is hydrogen and R2 is optionally substituted C1-C6 alkyl, phenyl or phenylmethyl, or R1 and R2 taken together with the C atom to which they are attached form a cyclopentyl, cyclohexyl or cycloheptyl ring.

29. The peptide of claim 26 wherein the helix inducing peptide comprises 4, 5 or 6 Lys residues.

30. The peptide of claim 26 which has an N-terminal Lys-Lys-Lys-Lys-Lys-Lys-Lys (SEQ ID NO:30) sequence.

31. (canceled)

32. The method of treatment of conditions responsive to activation of Y4 receptors the method comprising administering to a patient in need thereof an effective amount of a peptide as defined in claim 1.

33. The method of claim 32, wherein the condition treated is one for which regulation of energy intake or energy metabolism, control of intestinal secretion, decrease of gastrointestinal tract motility, or decrease of rate of gastric emptying, is indicated.

34. The of claim 33, wherein the condition treated is obesity or overweight, or a condition in which obesity or overweight is considered a contributory factor.

35. The method of claim 34 wherein the condition treated is Inflammatory bowel disease, bulimia, bulimia nervosa, Syndrome X (metabolic syndrome), diabetes, type 2 diabetes mellitus or Non Insulin Dependent Diabetes Mellitus (NIDDM), hyperglycemia, insulin resistance, impaired glucose tolerance, cardiovascular disease, hypertension, atherosclerosis, coronary artery disease, myocardial infarction, peripheral vascular disease stroke, thromboembolic diseases, hypercholesterolemia, hyperlipidemia, gallbladder disease, osteoarthritis, sleep apnea, reproductive disorders such as polycystic ovarian syndrome, or cancer of the breast, prostate, or colon.

36. The method of claim 34 wherein the agonist is administered to a patient in the fasted state.

37. The method of claim 32, wherein the condition treated is diarrhoea or hypersecretion from intestinal stomia.

38. The method of claim 32, wherein the condition treated is nausea or emesis.

39. The method of claim 38 wherein the condition of nausea or emesis treated is one arising from or anticipated to arise from treatment with another pharmaceutical agent.

40. The method of claim 31 wherein the Y4 selective receptor agonist comprises a GAG-binding motif.

41. The method of claim 31 wherein the Y4 selective receptor agonist comprises a serum-binding motif.

42. The method of claim 31 wherein the Y4 selective receptor agonist is PEGylated.

43. The method of claim 31, wherein the agonist is administered to a patient via a parenteral route including subcutaneous, intramuscular, intravenous, nasal, transdermal or buccal administration.
